(19) United States
(12) Patent Application Publication
(10) Pub. No.: US 2014/0370515 A1
(43) Pub. Date: Dec. 18, 2014

(54) METHOD OF CO-CULTURING HUMAN ENDOMETRIAL STEM CELLS AND RAT EMBRYONIC TOOTH BUD CELLS TO OBTAIN AMELOBLAST CELLS

(71) Applicants: Jafar Ai, Tehran (IR); Naghmeh Bahrami, Tehran (IR); Azadeh Babaee, TEHRAN (IR); Saeed Heydari, tehran (IR); Mina Jafar abadi, tehran (IR); Reza Rouzafzoun, TEHRAN (IR); Mohamah Hosein Asadi, TEHRAN (IR); Mohamad Tavangar, TEHRAN (IR); Amir Amani, TEHRAN (IR)

(72) Inventors: Jafar Ai, Tehran (IR); Naghmeh Bahrami, Tehran (IR); Azadeh Babaee, TEHRAN (IR); Saeed Heydari, tehran (IR); Mina Jafar abadi, tehran (IR); Reza Rouzafzoun, TEHRAN (IR); Mohamah Hosein Asadi, TEHRAN (IR); Mohamad Tavangar, TEHRAN (IR); Amir Amani, TEHRAN (IR)

(21) Appl. No.: 14/475,671

(22) Filed: Sep. 3, 2014

(51) Int. Cl. C12N 5/077

(52) U.S. Cl. CPC C12N 5/0654; C12N 2502/1364; C12N 2506/243; C12N 2501/155
USPC 435/6.12; 435/29; 435/39; 435/7.21

(57) ABSTRACT

The various embodiments herein provide a method of co-culturing human endometrial stem cells and rat embryonic tooth bud cells to obtain ameloblast cells. The human endometrial stem cells are isolated from human females between the ages of 18-40 and cultured. The hEnSCs are identified using flowcytometry. The rat embryonic tooth bud cells are isolated from rat embryos and cultured. After the hEnSCs and rat embryonic tooth bud cells reach a desired level of cell growth, the hEnSCs are transferred to culture plate inserts and the rat embryonic tooth bud cells are transferred to a culture plate. The hEnSCs and rat embryonic tooth bud cells are co-cultured to obtain ameloblast cells. Immunocytochemical analysis is used to characterize the differentiated ameloblast cells. Quantitative real time PCR (qRT-PCR) is used to detect the presence of Ameloblastin, Amelogenin, Amelotin and Cytokeratin 14 proteins in differentiated ameloblast cells.

### STATEMENT OF SPONSORSHIP

The present invention for international filling is sponsored by Tehran University of Medical Sciences

### BACKGROUND

1. Technical Field

The embodiments herein generally relate to field tissue engineering. The embodiments herein particularly relate tissue engineering for a repair and regeneration of teeth after damage, using biocompatible stem cells. The embodiments herein relate more particularly to a method of differentiation of human endometrial stem cells to ameloblast cells for teeth repair and regeneration.

2. Description of the Related Art

The human tooth is composed of complex biomaterials like dentin and enamel. Dentin makes up the bulk of all teeth. The dentin is harder than bone but softer than enamel. Enamel is the hardest tissue in vertebrates. The enamel protects the dentin from chewing, biting, crunching and even grinding. It also insulates the teeth from temperatures changes and harsh chemicals.

Tooth organogenesis is a result of reciprocal interactions between epithelial cells of the inner enamel organ and mesenchymal cells of the dental papilla. Odontoblasts are dental mesenchymal cells that give rise to the dentin of the teeth and ameloblast are epithelium-derived cells that form the enamel of the teeth. The reciprocal interaction between the ameloblast and odontoblast cells leads to their differentiation. The ameloblast and odontoblast cells deposit specialized mineralized matrices called enamel and dentin. Numerous signaling molecules, including BMPs, FGFs, WNTs and SHH are involved in different stages of the embryonic tooth development. Ameloblast cells undergo apoptosis after tooth eruption. The epithelially derived ameloblast stem cells are not present in a fully grown tooth. Therefore it is difficult to isolate ameloblast stem cells from the enamel of the tooth.

As a person gets older, the enamel gets damaged, become chipped or cracked. The conventional dental treatment makes use of synthetic materials to treat cavities, fill defects and even replace the whole tooth. A commonly used alternative method for tooth replacement is a dental implant.

A dental implant is a synthetic or artificial tooth that is placed in a person's jaw to replace a tooth, bridge between teeth and to fill a gap. Despite the long history of synthetic implants, there are several limitations in the functionality and longevity of the implants. Synthetic dental implants do not maintain the physiology and plasticity of naturally formed teeth. Therefore, these synthetic dental implants do not provide an ideal solution for tooth replacement.

Tissue engineering refers to the science of assembling biocompatible functional constructs that restore, maintain, and improve damaged tissues or whole organs. Regenerative medicine is a broad field that includes tissue engineering but also incorporates research on self-healing. In self-healing, the body uses its own natural biological system to repair or recreate cells, rebuild tissues and organs. Sometimes the foreign biological materials such as tissues or cells are administered to help the body recreate or repair its cells, rebuild tissues and organs.

Several experiments have been carried out in the field of tissue engineering and stem cell-based tissue regeneration. Studies are being carried out in the area of stem cell based regeneration and repair of cartilage, bones, tendons, muscle and adipose tissue. Differentiation of several dental epithelial cells except enamel cells has been successfully studied.

Experiments show that the mouse embryonic first arch epithelium cells, when co-cultured with non dental stem cells, including embryonic neural stem cells, adult bone marrow mesenchymal stem cells, and mouse embryonic first arch epithelium cells, induce the expression of dental mesenchyme molecular markers. However only 3 complete teeth are formed, When a co-cultured mouse first arch epithelial cells and bone marrow mesenchymal cells are transplanted into the adult mouth.

Ameloblast cells have been identified as a suitable cell source for cell replacement and regeneration of the enamel cells. Studies show that ameloblast cells and non dental mesenchymal stem cells, such as bone marrow stem cells, can undergo differentiation in the presence of dental mesenchymal stem cells to from a population of differentiated cells which can be used for cell therapy.

Recent studies indicate the mouse embryonic stem cells can undergo differentiation towards dental epithelial cell lineages, when the mouse embryonic stem cells are cultured in an ameloblast serum free conditioned medium (ASF-CM). But same results are not obtained, when similar experiments were carried out with the tooth germ cell-conditioned medium (TGC-CM).

Studies have also shown that the human keratinocytes cells are induced to express dental epithelial marker PITX2 and differentiated into enamel-secreting ameloblast cells, when human keratinocytes are combined with mouse embryonic dental mesenchyme in the presence of an exogenous growth factor-FGF8. The enamel secreting ameloblast cells develop a human-mouse chimeric whole tooth crown. The growth factor FGF8 plays an important role in the differentiation of keratinocytes into ameloblast cells. But the endogenous expression of FGF8 is never expressed in the dental mesenchyme and its expression ceases in the developing tooth around the bud stage in mice.

Human endometrial stem cells (hEnSCs) are an easily accessible source of adult stem cells. The human endometrial stem cells, like other mesenchymal lineage stem cells, have a low level of immunogenicity. Human endometrial stem cells are the most preferred type of cells arising from the mesenchymal stem cell lineage because of their high differentiation capacity, rapid growing and their functional development.

Recent researches indicate the presence of endometrial stem cells in different layers of the uterus. Immunohistochemistry has helped in the identification of specific markers such as Oct-4, CD146, and STRO-1 expressed by the endometrial stem cells. The endometrial stem cells can undergo differentiation, just like bone marrow stem cells and adipose derived stem cells.

Pig enamel cell culture studies show the use of a three dimensional Matrigel which induce the formation of epithelial cells. However, the Matrigel exhibits variations in different batches, and can introduce unwanted xenogeneic contaminants into the culture medium.

Some of the present stem cell culture methods make use of chemical stimulants for the differentiation of the stem cells. The use of chemical stimulants for the purpose of stem cell differentiation is still limited by the quality, source, and amount of the utilized reagents.

Although several experiments have been carried out in the area of dental tissue engineering and regeneration using stem cells, the studies should focus on the ways/methods to reactivate odontogenic program using stem cells and to formulate consistent, stable and efficient protocols for tooth organogenesis or regeneration.

Hence there is a need to develop a method for differentiating human endometrial stem cells to obtain ameloblast cells by using a co-culturing technique. Further there is a need to develop a three dimensional culture system without the use of chemical stimulants for the differentiation of human endometrial stem cells.

The above mentioned shortcomings, disadvantages and problems are addressed herein and which will be understood by reading and studying the following specification.

### OBJECTIVES OF THE EMBODIMENTS

The primary object of the embodiments herein is to provide a co-culture system for the differentiation of rat embryonic tooth bud and human endometrial stem cells (hEnSCs) to obtain the ameloblast cells.

Another object of the embodiments herein is to provide a non-contact co-culture system for the differentiation of rat embryonic tooth bud cell and human endometrial stem cells to obtain the ameloblast cells.

Yet another object of the embodiments herein is to provide a co-culture system using culture plate inserts for the differentiation of rat embryonic tooth bud cells and human endometrial stem cells to obtain the ameloblast cells.

Yet another object of the embodiments herein is to provide a three dimensional co-culture system free of chemical stimulants for the differentiation of the ameloblast cells.

Yet another object of the embodiments herein is to provide a suitable micro-environment to regulate and maintain the differentiation of human endometrial stem cells to obtain the ameloblast cells.

Yet another object of the embodiments herein is to provide a co-culture system with the appropriate combination of stem cell population with adequate signaling molecules.

Yet another object of the embodiments herein is to provide a population of endometrial stem cells that expresses CD29, CD44, CD90, CD146 and CD105 markers in DMEM based culture medium.

Yet another object of the embodiments herein is to provide a differentiating stem cell population capable of stimulating angiogenesis.

Yet another object of the embodiments herein is to provide a differentiating stem cell population capable of stimulating the differentiation of endogenous and exogenous progenitor cells.

Yet another object of the embodiments herein is to provide a differentiating stem cell population capable of decreasing, preventing, blocking, limiting and controlling T cell mediated immune response in vitro and in vivo.

Yet another object of the embodiments herein is to provide biocompatible cell sources with a low level of immunogenicity for the clinical regeneration of the ameloblast cells.

Yet another object of the embodiments herein is to provide cell bank with differentiated ameloblast cells for commercialization purposes.

Yet another object of the embodiments herein is to provide cell bank with differentiated ameloblast cells for the treatment of dental diseases, tissue regeneration and tissue repair.

These and other objects and advantages of the embodiments herein will become readily apparent from the following detailed description taken in conjunction with the accompanying drawings.

### SUMMARY

The various embodiments herein provide a method for the differentiation of human endometrial stem cells (hEnSCs) to ameloblast cells by culturing hEnSCs with rat embryonic tooth mesenchyme cells with growth factor fibroblast growth factor 8 (FGF8). Also the ameloblast cells are used as natural product in dental tissue engineering. The hEnSCs give rise to different kinds of epithelial cells such as odontoblast cells. This indicates the potential importance of hEnSCs to serve as a source for ameloblasts. The hEnSCs are reprogrammed to give rise to ameloblast cells, thereby offering novel potentials for tooth-tissue engineering. The ameloblast cells find their application in the treatment of the neurodegerative disorder, liver dysfunction and dental tissue reconstruction.

According to one embodiment herein, a method of synthesizing ameloblast cells comprises the following major steps. The human endometrial stem cells (hEnSCs) are isolated from human female patients and cultured. The human endometrial stem cells are identified using flow cytometry. The rat tooth bud stem cells are isolated from rat embryos and cultured. The human endometrial stem cells are co-cultured with the rat embryonic tooth bud cells to obtain differentiated ameloblast cells. The differentiated ameloblast cells are characterized.

According to one embodiment herein, the method of isolating and culturing human endometrial stem cells (hEnSCs) comprises the steps of acquiring a plurality of endometrial biopsy samples from female patients of preset age group, and wherein the female patients are in the age groups of 18-40. The endometrial biopsy samples are transferred into Hanks fluid. Further the endometrial biopsy samples are washed in a phospahte buffer saline (PBS) containing antibiotics. The endometrial biopsy samples are sliced into small fragments. The small fragments are immersed into a proteolytic enzymes and DMEM containing antibiotics for two hours at 37° C. The proteolyitc enzyme is selected from a group consisting of a collagenase IA and a trypsin. The epithelial stem cells and the stromal cells present in the endometrial biopsy samples are separated using a plurality of filters. The plurality of filters has a size of 45 μm and 70 μm. The separated cells are centrifuged at 1000 rpm for 15 minutes and the cells are purified by a Ficoll purification. The purified cells are placed in a flask and the purified cells are incubated at 37° C. in an atmosphere containing 5% CO2 and 95% moisture. The purified cells are analyzed using a flow cytometry.

According to one embodiment herein, the step of isolating and culturing the rat embryonic tooth bud cells comprises the step of acquiring a plurality of Wistar rat embryos from an anaesthetized pregnant female rat. The acquired rat embryos are in an age group of 7 days, 9 days, 10 days, 12 days and 14 days. The rat mesenchymal tooth bud tissue is isolated from a lower jaw of the rat embryo using a scalpel. The embryonic tooth bud tissue is trypsinized by placing the tooth bud tissue in 1% trypsin at 4° C. The rat embryo tooth bud tissue trypsinization yields dental epithelial cells and mesenchymal cells. The dental epithelium cells and mesenchymal cells are separated under a stereo microscope. The dental epithelial cells and the mesenchymal cells are rinsed in phosphate buffer saline (PBS). The dental epithelial cells and the mesenchymal cells are subjected to an enzyme for 3 hours at 37° C. The dental epithelial cells and the mesenchymal cells are filtered using a nylon filter with a pore size of 70 mm and a filtrate is obtained. The filtrate is centrifuged at 2000 g for 15 minutes and rinsing the centrifuged filtrate with phosphate buffer saline. The dental epithelial cells and mesenchymal cells are transferred to a culture plate containing 0.5 mL DMEM culture medium and the DMEM culture medium further comprises 100× antibiotic, 10% FBS and 1% gentamycine. The culture plate is a 6 well culture plate for culturing the cells. The 6 well culture plate is incubated in an atmosphere containing 5% CO2, and 95% humidity for two weeks. The growth medium is replaced once every 72 hours.

According to one embodiment herein, the enzyme for the dental epithelium treatment is selected from a group consisting of collagenase IA, trypsin, dispase and a chelating agent and the chelating agent comprises 10 mM EDTA with Ca2+, Mg2+, and PBS.

According to one embodiment herein, the rat mesenchymal tooth bud tissues with different ages are treated with different chelating agent or enzyme, and wherein the 7 days old rat mesenchymal tooth bud tissue is treated with a chelating agent comprising 10 mM EDTA containing Ca2+, Mg2+ and PBS. The 9 days old rat mesenchymal tooth bud tissue is treated with an enzyme dispase. The 10 days old rat mesenchymal tooth bud tissue is treated with an enzyme trypsin. The 12 days old rat mesenchymal tooth bud tissue is treated with an enzyme collagenase IA. The 14 days old rat mesenchymal tooth bud tissue is treated with an enzyme trypsin.

According to one embodiment herein, the steps of co-culturing the human endometrial stem cells (hEnSCs) with the rat embryonic tooth bud cells to obtain a differentiated ameloblast cells is initiated with culturing the human endometrial stem cells (hEnSCs) for three passages. After three passages, the hEnScs reach a desired cell density of 1×106 cells/ml. The hEnSCs are transferred to a culture plate inserts of the 6 well culture plate. The culture plate insert comprises 2 mL DMEM with 10% FBS culture medium. The rat mesenchymal tooth bud cells are cultured for two passages, and the rat mesenchymal tooth bud cells are cultured for obtaining a cell density of 1×105 cells/ml. The rat mesenchymal tooth bud cells are transferred to the 6 well culture plate. Further the culture insert with hEnSCs is put in the culture plate containing rat mesenchymal tooth bud cells. A growth factor agent is added to the environment surrounding the endometrial cells. The hEnSCs and the rat mesenchymal tooth bud cells are counted using a neobar lam cell counter. The endometrial stem cells are treated with differentiation medium. The medium is changed once in every 72 hours.

According to one embodiment herein, the growth factor agent surrounding the endometrial cells is selected from a group consisting of a Fgf8, a BMP2 and a BMP4.

According to one embodiment herein, the rat mesenchymal tooth bud cells of different ages are co-cultured with the human endometrial stem cells (hEnSCs) supplemented with different growth factor agents. The rat embryonic mesenchymal tooth bud cells of 14 days old are co-cultured with the human endometrial stem cells (hEnSCs) supplemented with a growth factor agent of Fgf8. The rat embryonic mesenchymal tooth bud cells of 7 days old are co-cultured with the human endometrial stem cells (hEnSCs) supplemented with a growth factor agent of BMP2. The rat embryonic mesenchymal tooth bud cells of 9 days old are co-cultured with the human endometrial stem cells (hEnSCs) supplemented with a growth factor agent of BMP4. The rat embryonic mesenchymal tooth bud cells of 10 days old are co-cultured with the human endometrial stem cells (hEnSCs) supplemented with a growth factor agent of Fgf8. The rat embryonic mesenchymal tooth bud cells of 12 days old are co-cultured with the human endometrial stem cells (hEnSCs) supplemented with a growth factor agent of Fgf8.

According to one embodiment herein, the human endometrial endometrial stem cells (hEnSCs) are accessible source of adult stem cells. The human endometrial endometrial stem cells (hEnSCs) are like other mesenchymal lineage stem cells with a low level of immunogenicity. The hENSCs are favourable cells of the mesenchymal stem cell lineage because of high differentiation capacity, rapid growing and their functional development characteristics/properties.

According to one embodiment herein, the interaction of two types of cells is used to differentiate into ameloblast cells. The most important issue in the regulation and maintenance of cells is their micro-environments. It is believed that the micro-environmental elements intrinsically exist in natural niche of stem cells to regulate the cells fate and provide necessary elements for the cell population. The xeno-free non-contact co-culture or culture inserts method is used To stimulate these events in vitro and avoiding the use of chemical agents for proper ameloblast differentiation as much as possible. The number of differentiated cells and a quality of differentiation is highly important and noticeable in the cell culture of dental tissue engineering.

According to one embodiment herein, the method for a regeneration and differentiation of ameloblast cells from the human endometrial stem cells and embryonic tooth stem cells comprises stem cell isolation and identification of the isolated stem cells, ameloblast differentiation by culture insert method and characterization of the ameloblast stem cells.

According to one embodiment herein, the human endometrial stem cells (hENSCs) and mouse tooth bud epithelial cells are isolated. The isolated cells are subjected for identification. The isolated cells are identified by flowcytometry, or IHC, or qRT-PCR. The isolated stem cells are subjected to ameloblast differentiation by a culture insert method. The hENSCs along with the epithelial cells of embryonic mouse tooth bud differentiate into ameloblast cells. After the differentiation of the cells into ameloblast cells, the ameloblast cells are subjected to characterization. The characterization of the cells is done by immunohistochemistry (IHC), or real-time PCR.

According to one embodiment herein, the endometrial stem cells are obtained from the ten biopsy samples of the female patients aged between 18-40 years of the childbearing ages. The samples are transferred to the cell culture laboratory in the hanks fluid. The endometrial biopsy samples are washed in the phosphate-buffered saline solution containing antiniotics, sliced into small fragments, immersed in collagenase (IA) (2 mg/ml in DMEM containing antibiotics) and incubated for 2 hours at 37° C. The epithelial cells and the stromal cells are separated using filters with a size of 45 μm and 70 μm. After passing through the filters, the filtrate is centrifuged at 1000 rpm for 15 minutes and then ficoll purification is performed. The flask is incubated at 37° C., in an atmosphere containing 5% CO2 and 95% moisture. In order to analyze the net stem cells material using a flow cytometry technique, the net cell suspension with a concentration of 1×107 cells/ml is treated for one hour at 4° C. with antibodies comprising CD90, anti CD 105, FITC-phycoerythrin (PE), anti-CD 34, anti-human 31, or PE-conjugated mouse IgG1, anti-CD61, and anti-CD 14. The cells treated with antibodies are incubated at 37° C., in atmosphere containing 5% CO2 and 95% moisture for 24 hours. The incubated cells are washed with PBS and are prepared for flow cytometry (Partec, Germany).

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart explaining the various steps of isolating, identifying, co-culturing and characterizing the hEnSCs and rat embryonic tooth bud cells to obtain differentiated ameloblast cells, according to one embodiment herein.

FIG. 2 illustrates a flow chart indicating the various steps involved in co-culturing the hEnSCs and rat embryonic tooth bud cells to obtain ameloblast cells, according to one embodiment herein.

FIG. 3 illustrates a histogram indicating a relative gene expression of ameloblast specific mRNAs CK14, AMTN, AMGN and AMBN in differentiated and undifferentiated hEnSCs at day 14 of induction, according to one embodiment herein.

FIG. 4 illustrates a histogram indicating a flow cytometry analysis of endometrial cells for mesenchymal stem cell marker CD90 and hematopoietic stem cell marker CD34, according to one embodiment herein.

FIG. 5A-5C illustrate the images indicating the morphological characteristics of human endometrial stem cells before and after differentiation, according to one embodiment herein.

FIG. 6A-6C illustrate the images indicating the morphological characteristics of differentiated human endometrial stem cells (hEnSCs) after co-culturing the hEnSCs with rat embryo dental tooth bud stem cells, according to one embodiment herein.

FIG. 7 illustrates an image indicating the mineralized nodule stained with Alizarin red stain, according to one embodiment herein.

FIG. 8A-8D illustrate the images indicating the immunocytochemical analysis of Ameloblastin markers, in the differentiated human endometrial stem cells (hEnSCs), according to one embodiment herein.

FIG. 9A-9D illustrate the images indicating the immunocytochemical analysis of Amelogenin markers, in the differentiated human endometrial stem cells (hEnSCs), according to one embodiment herein.

FIG. 10 illustrates a schematic representation of a non-contact co-culture system of the human endometrial stem cells (hEnSCs) rat embryonic tooth bud cells, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The following detailed description uses certain abbreviations. The following is a list of full names of the abbreviations used to describe the various embodiments herein:
hEnSCs: Human Endometrial Stem
PCR: Polymerase Chain Reaction
FGF8: Fibroblast Growth Factor 8
BMP2: Bone morphogenetic protein 2
BMP4: Bone morphogenetic protein 4
CD34: cell surface glycoprotein expressed on primitive hematopoietic stem cells
CD90: cell surface glycoprotein expressed in many cell types such as neurons, thymocytes, fibroblasts, endothelial cells.
CD105: type I membrane glycoprotein located on cell surfaces and is part of the TGF beta receptor complex.
CD31: Platelet endothelial cell adhesion molecule found on the surface of platelets, monocytes, neutrophils, endothelial cells and some types of T-cells.
CD61: membrane glycoprotein expressed on the surface of platelets and megakariocytes
CD14: cell surface glycoprotein strongly expressed on most human monocytes and macrophages in peripheral blood, other bodily fluids, and in various tissues such as lymph nodes and spleen.
DMEM: Dulbecco's Modified Eagle Medium
DMSO: Dimethyl sulfoxide
Collagenase IA: collagenase from Clostridium histolyticum
Dispase: Dispase is a protease which cleaves fibronectin, collagen IV, and to a lesser extent collagen I.
rpm: revolutions per minute
FITC: Fluorescein isothiocyanate
PE: Phycoerythrin
IgG1: Immunoglobulin G subclass 1
PBS: phosphate buffer saline
FBS: Fetal bovine serum
ARS: Alizarin red staining
RNX-Plus: Guanidine/phenol solution for total RNA isolation
RNeasy Plus: mini kit for purification of high-quality RNA from cells, tissues, and yeast using silica-membrane.
RPMI: Roswell Park Memorial Institute medium
DEPC: Diethylpyrocarbonate is used in the laboratory to inactivate RNase enzymes in water.
PCR: Polymerase chain reaction
qRT-PCR: Quantitative real time PCR
AMTN: Amelotin
AMGN: Amelogenin
AMBN: Ameloblastin
CK14: Cytokeratin 14
FBS: Fetal bovine serum is the most widely used serum-supplement for in vitro cell culture of eukaryotic cells.
FCS: Fetal calf serum also known as Fetal Bovine Serum.
HSA: Human serum albumin is the most abundant protein in human blood plasma and constitutes about half of the blood serum protein.

The following are the list of all materials, reagents, company and company code used in the embodiment.

| Code No | Company | Material |
|---|---|---|
| 667382 | Sigma | Tris (2,4-dimethyl-5-sulfophenyl) |
| | | phosphine trisodium salt |
| 352340 | Falcon | 40 micro m cell strainer |
| 352350 | Falcon | 70 micro m cell strainer |
| K-2044 | Bioneer | AccuPower ® RocketScript ™ |
| | | RT PreMix kit |
| h1758 | Sigma | HCl |
| a5533 | Sigma | Alizarin red |
| APF | Sigma | ALP assay kit |
| ab72776 | Abcam | Ameloblastin antibody |
| ab59705 | Abcam | Amelogenin antibody |
| A2942 | Sigma | Amphitericin B |
| 420042 | Sigma | Mgcl₂ |
| A2153 | Sigma | BSA |
| ab11415 | Abcam | CD105-antibody |
| orb10288 | Biorbyt | CD133-antibody |
| ab11415 | Abcam | CD146-antibody |
| 17100-017 | Invitrogen | Collagenase, type I |
| C9811-12EA | Sigma | CryoCane ™ |
| 21885025 | Invitrogen | DMEM low glucose with L-glutamine |
| | | (1 g/1) 500 mL |
| 31331028 | Invitrogen | DMEM/F-12 |
| d8418 | Sigma | DMSO |
| 10270106 | Invitrogen | FBS |
| F0291 | Sigma | FGF8 |
| 14170112 | Sigma | Hank's balanced salt solution 500 mL |
| H6136 | Sigma | Hanks media |
| 30412 | Sigma | HNa₂PO₄ |
| 25030-024 | Invitrogen | L-Glutamine |
| 000-10 | Yazd syringe | Syringe 10 cc |
| 000-05 | Yazd syringe | Syringe 5 cc |
| 000-02 | Yazd syringe | Syringe 2 cc |
| 204447 | Sigma | Sodium sulfate |
| 158127 | Sigma | paraformaldehyde |
| 421000013 | Sigma | Pipette |
| p7626 | Sigma | PMSF |
| 74136 | Qiagen | RNeasy ® PLUS mini kit |
| 59417C | Sigma | Trypsin/EDTA |
| BD | Sigma | Tube (15 mL) |
| BD | Sigma | Tube (50 mL) |
| 90075 | Sigma | Flask T25 |
| 0002 | Sigma | Nitrate calcium |
| c1016 | Sigma | Cacl2 |
| 15596-026 | Invitrogen | TRIzol ® Reagent |
| 204143 | Qiagen | QuantiTect SYBR Green PCR Kit |
| K-2101 | Bioneer | AccuPower ® RocketScrpt ™ |
| | | RT PreMix |
| s3014 | Sigma | NaCl₂ |
| ab5758 | Abcam | Rabbit F(ab′) 2 Anti-Mouse IgG-H&L |
| | | (FITC), pre-adsorbed |
| | HBG | Neobar lam cell counter |
| | (Germany) | |
| 14-9161-71 | eBioscience | Human Fc Receptor Binding |
| | (Affymetrix) | Inhibitor Purified |
| D4818 | Sigma | Dispase I |

The various embodiments herein provide a method for the differentiation of human endometrial stem cells (hEnSCs) to ameloblast cells by culturing hEnSCs with rat embryonic tooth mesenchyme cells with growth factor fibroblast growth factor 8 (FGF8). Also the ameloblast cells are used as natural product in dental tissue engineering. The hEnSCs give rise to different kinds of epithelial cells such as odontoblast cells. This indicates the potential importance of hEnSCs to serve as a source for ameloblasts. The hEnSCs are reprogrammed to give rise to ameloblast cells, thereby offering novel potentials for tooth-tissue engineering. The ameloblast cells find their application in the treatment of the neurodegerative disorder, liver dysfunction and dental tissue reconstruction.

According to one embodiment herein, a method of synthesizing ameloblast cells comprises the following major steps. The human endometrial stem cells (hEnSCs) are isolated from human female patients and cultured. The human endometrial stem cells are identified using flow cytometry. The rat tooth bud stem cells are isolated from rat embryos and cultured. The human endometrial stem cells are co-cultured with the rat embryonic tooth bud cells to obtain differentiated ameloblast cells. The differentiated ameloblast cells are characterized.

According to one embodiment herein, the method of isolating and culturing human endometrial stem cells (hEnSCs) comprises the steps of acquiring a plurality of endometrial biopsy samples from female patients of preset age group, and wherein the female patients are in the age groups of 18-40. The endometrial biopsy samples are transferred into Hanks fluid. Further the endometrial biopsy samples are washed in a phospahte buffer saline (PBS) containing antibiotics. The endometrial biopsy samples are sliced into small fragments. The small fragments are immersed into a proteolytic enzymes and DMEM containing antibiotics for two hours at 37° C. The proteolyitc enzyme is selected from a group consisting of a collagenase IA and a trypsin. The epithelial stem cells and the stromal cells present in the endometrial biopsy samples are separated using a plurality of filters. The plurality of filters has a size of 45 μm and 70 μm. The separated cells are centrifuged at 1000 rpm for 15 minutes and the cells are purified by a Ficoll purification. The purified cells are placed in a flask and the purified cells are incubated at 37° C. in an atmosphere containing 5% CO2 and 95% moisture. The purified cells are analyzed using a flow cytometry.

According to one embodiment herein, the step of isolating and culturing the rat embryonic tooth bud cells comprises the step of acquiring a plurality of Wistar rat embryos from an anaesthetized pregnant female rat. The acquired rat embryos are in an age group of 7 days, 9 days, 10 days, 12 days and 14 days. The rat mesenchymal tooth bud tissue is isolated from a lower jaw of the rat embryo using a scalpel. The embryonic tooth bud tissue is trypsinized by placing the tooth bud tissue in 1% trypsin at 4° C. The rat embryo tooth bud tissue trypsinization yields dental epithelial cells and mesenchymal cells. The dental epithelium cells and mesenchymal cells are separated under a stereo microscope. The dental epithelial cells and the mesenchymal cells are rinsed in phosphate buffer saline (PBS). The dental epithelial cells and the mesenchymal cells are subjected to an enzyme for 3 hours at 37° C. The dental epithelial cells and the mesenchymal cells are filtered using a nylon filter with a pore size of 70 mm and a filtrate is obtained. The filtrate is centrifuged at 2000 g for 15 minutes and rinsing the centrifuged filtrate with phosphate buffer saline. The dental epithelial cells and mesenchymal cells are transferred to a culture plate containing 0.5 mL DMEM culture medium and the DMEM culture medium further comprises 100× antibiotic, 10% FBS and 1% gentamycine. The culture plate is a 6 well culture plate for culturing the cells. The 6 well culture plate is incubated in an atmosphere containing 5% CO2, and 95% humidity for two weeks. The growth medium is replaced once every 72 hours.

According to one embodiment herein, the enzyme for the dental epithelium treatment is selected from a group consisting of collagenase IA, trypsin, dispase and a chelating agent and the chelating agent comprises 10 mM EDTA with Ca2+, Mg2+, and PBS.

According to one embodiment herein, the rat mesenchymal tooth bud tissues with different ages are treated with different chelating agent or enzyme, and wherein the 7 days old rat mesenchymal tooth bud tissue is treated with a chelating agent comprising 10 mM EDTA containing Ca2+, Mg2+ and PBS. The 9 days old rat mesenchymal tooth bud tissue is treated with an enzyme dispase. The 10 days old rat mesenchymal tooth bud tissue is treated with an enzyme trypsin. The 12 days old rat mesenchymal tooth bud tissue is treated with an enzyme collagenase IA. The 14 days old rat mesenchymal tooth bud tissue is treated with an enzyme trypsin.

According to one embodiment herein, the steps of co-culturing the human endometrial stem cells (hEnSCs) with the rat embryonic tooth bud cells to obtain a differentiated ameloblast cells is initiated with culturing the human endometrial stem cells (hEnSCs) for three passages. After three passages, the hEnScs reach a desired cell density of 1×106 cells/ml. The hEnSCs are transferred to a culture plate inserts of the 6 well culture plate. The culture plate insert comprises 2 mL DMEM with 10% FBS culture medium. The rat mesenchymal tooth bud cells are cultured for two passages, and the rat mesenchymal tooth bud cells are cultured for obtaining a cell density of 1×105 cells/ml. The rat mesenchymal tooth bud cells are transferred to the 6 well culture plate. Further the culture insert with hEnSCs is put in the culture plate containing rat mesenchymal tooth bud cells. A growth factor agent is added to the environment surrounding the endometrial cells. The hEnSCs and the rat mesenchymal tooth bud cells are counted using a neobar lam cell counter. The endometrial stem cells are treated with differentiation medium. The medium is changed once in every 72 hours.

According to one embodiment herein, the growth factor agent surrounding the endometrial cells is selected from a group consisting of a Fgf8, a BMP2 and a BMP4.

According to one embodiment herein, the rat mesenchymal tooth bud cells of different ages are co-cultured with the human endometrial stem cells (hEnSCs) supplemented with different growth factor agents. The rat embryonic mesenchymal tooth bud cells of 14 days old are co-cultured with the human endometrial stem cells (hEnSCs) supplemented with a growth factor agent of Fgf8. The rat embryonic mesenchymal tooth bud cells of 7 days old are co-cultured with the human endometrial stem cells (hEnSCs) supplemented with a growth factor agent of BMP2. The rat embryonic mesenchymal tooth bud cells of 9 days old are co-cultured with the human endometrial stem cells (hEnSCs) supplemented with a growth factor agent of BMP4. The rat embryonic mesenchymal tooth bud cells of 10 days old are co-cultured with the human endometrial stem cells (hEnSCs) supplemented with a growth factor agent of Fgf8. The rat embryonic mesenchymal tooth bud cells of 12 days old are co-cultured with the human endometrial stem cells (hEnSCs) supplemented with a growth factor agent of Fgf8.

According to one embodiment herein, the human endometrial endometrial stem cells (hEnSCs) are accessible source of adult stem cells. The human endometrial endometrial stem cells (hEnSCs) are like other mesenchymal lineage stem cells with a low level of immunogenicity. The hENSCs are favourable cells of the mesenchymal stem cell lineage because of high differentiation capacity, rapid growing and their functional development characteristics/properties.

According to one embodiment herein, the interaction of two types of cells is used to differentiate into ameloblast cells. The most important issue in the regulation and maintenance of cells is their micro-environments. It is believed that the micro-environmental elements intrinsically exist in natural niche of stem cells to regulate the cells fate and provide necessary elements for the cell population. The xeno-free non-contact co-culture or culture inserts method is used To stimulate these events in vitro and avoiding the use of chemical agents for proper ameloblast differentiation as much as possible. The number of differentiated cells and a quality of differentiation is highly important and noticeable in the cell culture of dental tissue engineering.

According to one embodiment herein, the method for a regeneration and differentiation of ameloblast cells from the human endometrial stem cells and embryonic tooth stem cells comprises stem cell isolation and identification of the isolated stem cells, ameloblast differentiation by culture insert method and characterization of the ameloblast stem cells.

According to one embodiment herein, the human endometrial stem cells (hENSCs) and mouse tooth bud epithelial cells are isolated. The isolated cells are subjected for identification. The isolated cells are identified by flowcytometry, or IHC, or qRT-PCR. The isolated stem cells are subjected to ameloblast differentiation by a culture insert method. The hENSCs along with the epithelial cells of embryonic mouse tooth bud differentiate into ameloblast cells. After the differentiation of the cells into ameloblast cells, the ameloblast cells are subjected to characterization. The characterization of the cells is done by immunohistochemistry (IHC), or real-time PCR.

According to one embodiment herein, the endometrial stem cells are obtained from the ten biopsy samples of the female patients aged between 18-40 years of the childbearing ages. The samples are transferred to the cell culture laboratory in the hanks fluid. The endometrial biopsy samples are washed in the phosphate-buffered saline solution containing antiniotics, sliced into small fragments, immersed in collagenase (IA) (2 mg/ml in DMEM containing antibiotics) and incubated for 2 hours at 37° C. The epithelial cells and the stromal cells are separated using filters with a size of 45 μm and 70 μm. After passing through the filters, the filtrate is centrifuged at 1000 rpm for 15 minutes and then ficoll purification is performed. The flask is incubated at 37° C., in an atmosphere containing 5% CO2 and 95% moisture. In order to analyze the net stem cells material using a flow cytometry technique, the net cell suspension with a concentration of 1×107 cells/ml is treated for one hour at 4° C. with antibodies comprising CD90, anti CD 105, FITC-phycoerythrin (PE), anti-CD 34, anti-human 31, or PE-conjugated mouse IgG1, anti-CD61, and anti-CD 14. The cells treated with antibodies are incubated at 37° C., in atmosphere containing 5% CO2 and 95% moisture for 24 hours. The incubated cells are washed with PBS and are prepared for flow cytometry (Partec, Germany).

According to one embodiment herein, the rat tooth bud mesenchymal stem cells are obtained from eleven Winstar rat embryos. The 14 days old rat embryos are obtained from a pregnant female rat which is anesthetized. The lower jaw tooth bud embryos are removed by scalpel. The tooth buds are incubated for 2-3 hours in 1% trypsin at 4° C. The dental epithelium cells and mesenchymal stem cells are separated, after the enzymatic digestion of the tooth bud under the stereo microscope. The Phosphate-buffered saline is then used to rinse the separated mesenchymal stem cells and the rinsed mesenchymal stem cells are exposed to collagenase IA for 3 hours at 37° C. Then the exposed stem cells are passed through the 70 Mm Nylon filter. The filtrate cell samples are centrifuged by 2000 g for 15 min and rinsed again with phosphate buffer saline (PBS). The cells are transferred to 6 well plate containing 0.5 ml usual culture medium DMEM (antibiotic 100×, FBS 10% and Glutamin 1%) and incubated in an atmosphere containing 5% CO₂ and 95% humidity. The cells are maintained in the incubated condition for 1 week to achieve the desired density. The growth medium is replaced once in every 72 hours.

According to one embodiment herein, the rat tooth bud mesenchymal stem cells are obtained from eleven Winstar rat embryos. The 7 days old rat embryos are obtained from a pregnant female rat which is anesthetized. The embryo lower jaw tooth bud is removed by scalpel. The tooth buds are kept in 10 mM EDTA in Ca⁺⁺ and Mg⁺⁺ free phosphate buffer saline (PBS) at 37° C. for 2-3 hours. After the enzymatic digestion, the dental epithelium and the mesenchyme are separated under the stereo microscope. The Phosphate-buffered saline is then used to rinse the separated mesenchymal stem cells and the rinsed mesenchymal stem cells are exposed to collagensase IA for 3 hour at 37° C. Next, the exposed stem cells are filtered through Nylon filter 70 Mm. The filtered cell samples are then centrifuged by 1500 rpm for 15 minutes. After centrifugation, the cells are rinsed with phosphate buffer saline (PBS). The cells are then transferred to 6 well plate containing 0.5 ml usual culture medium DMEM (antibiotic 100×, FBS 10% and Glutamin 1%) and incubated in an atmosphere containing 5% CO₂ and 95% humidity. The incubated cells are maintained for 1 week to achieve the desired density. The growth medium is replaced once every 72 hours.

According to one embodiment herein, the rat tooth bud mesenchymal stem cells are obtained from eleven Winstar rat embryos. The 9 days old rat embryos are obtained from a pregnant female rat which is anesthetized. The embryo lower jaw tooth bud are removed by scalpel. The tooth buds are kept at 4° C. for 2-3 hours in enzyme Dispase. After the enzymatic treatment, the dental epithelium and mesenchymal cells are separated under stereo microscope. The separated cells are rinsed with phosphate buffer saline (PBS) and exposed to collagenase IA for 6 hours at 37° C. After the enzymatic digestion, the exposed cells are passed through 70 Mm Nylon filter. The filtrate is centrifuged at 2000 rpm for 15 minutes. After centrifugation, the cells are rinsed with PBS. The cells are then transferred to a 6 well plate containing 0.5 ml culture medium RPMI (antibiotic 100×, FBS 10% and glutamine 1%). The culture plate is then incubated at an atmosphere containing 5% CO₂ and 95% humidity. The incubated cells are maintained for 1 week to achieve the desired density. The growth medium is then replaced once every 72 hours.

According to one embodiment herein, the rat tooth bud mesenchymal stem cells are obtained from eleven Winstar rat embryos. The 10 days old rat embryos are obtained from a pregnant female rat which is anesthetized. The embryo lower jaw tooth bud is removed by scalpel. The tooth buds are kept at 4° C. for 2-3 hours in 1% trypsin. After the enzymatic treatment, the dental epithelium and mesenchymal cells are separated under stereo microscope. The separated cells are rinsed with phosphate buffer saline (PBS) and exposed to collagenase IA for 3 hours at 37° C. After the enzymatic digestion the cells are passed through 70 Mm Nylon filter. The filtrate is centrifuged at 1000 rpm for 5 minutes. After centrifugation, the cells are rinsed with PBS. The rinsed cells are then transferred to a 6 well plate containing 0.5 ml culture medium RPMI (antibiotic 100×, FBS 10% and glutamine 1%). The culture plate is then incubated at an atmosphere containing 5% CO₂ and 95% humidity. The incubated cells are maintained for 1 week to achieve the desired density. The growth medium is then replaced once every 72 hours.

According to one embodiment herein, the rat tooth bud mesenchymal stem cells are obtained from eleven Winstar rat embryos. The 12 days old rat embryos are obtained from a pregnant female rat which is anesthetized. The embryo lower jaw tooth bud is removed by scalpel. The tooth buds are kept at 4° C. for 2-3 hours in collagenase IA. After the enzymatic treatment, the dental epithelium and mesenchymal cells are separated under stereo microscope. The cells are rinsed with phosphate buffer saline (PBS) and exposed to Dispase for 3 hours at 37° C. After the enzymatic digestion, the cells are passed through 70 Mm Nylon filter. The filtrate is centrifuged at 2000 rpm for 15 minutes. After centrifugation, the cells are rinsed with PBS. The centrifuged cells are then transferred to a 6 well plate containing 0.5 ml culture medium DMEM (antibiotic 100×, FBS 10% and glutamine 1%). The culture plate is then incubated at an atmosphere containing 5% CO₂ and 95% humidity. The incubated cells are maintained for 1 week to achieve the desired density. The growth medium is then replaced once every 72 hours.

According to one embodiment herein, the human endometrial stem cells (hEnSCs) and the rat embryo tooth bud stem cells are co-cultured. After the third passage, the endometrial cells with a density of 1×10⁶ cell/ml is transferred to the culture plate insert of the 6 wells culture plate containing 2 ml DMEM supplemented with 10% FBS culture medium. The mesenchymal tooth bud stem cells with a density of 1×10⁵ cell/ml are transferred to the 6 well culture plates, after the second passage. After 24 hours, a fibroblast growth factor [FGF (100 ng/mv)] or bone morphogenetic protein (BMP2) are added to the environment containing the two adjacent cells of endometrial stem cells. The medium is changed once in every 72 hours. The counting of the cells is performed by Neonar lam (HBG, Germany). The endometrial cells are treated for 14 days with differentiation medium. The medium is changed once in every 72 hours.

According to one embodiment herein, the human endometrial stem cells (hEnSCs) and the rat embryo tooth bud stem cells are co-cultured to differentiate the co-culture into ameloblast cells. The differentiated cells obtained are characterized by analyzing the morphology, Alizarin red staining, Immunocytochemistry and quantitative real time PCR (qRT-PCR).

According to one embodiment herein, the morphology of the stem cells differentiated into ameloblast cells is analyzed regularly by observing the cell appearance, organization and the morphological changes. The differentiated ameloblast are analyzed using inverted microscope (Nikon, Japan TS-10).

According to one embodiment herein, the mineralization nodule and calcification of the stem cells differentiated into ameloblast cells is analyzed by Alizarin red staining. The Alizarin red staining for analysing mineralization nodule formation and calcification is performed after 28 days. The cells are washed three times with phosphate buffer saline (PBS). After washing the cells with PBS, the cells are fixed in 4% para-formaldehyde for 30 minutes. After 30 minutes, the cells are washed once with PBS. The staining is performed with 2% Alizarin red stain at a pH 4.2-4.4 for 30 minutes at 37° C. The stained areas are observed and pictured with inverted microscope.

According to one embodiment herein, the immuno-histochemistry test is done for the stem cells differentiated into ameloblast cells. The antibodies amelogenin (rabbit monoclonal anti-human) and ameloblastin (rabbit monoclonal anti-human) are used for determining stem cells differentiated to ameloblast cells at 14 days after the culture. The cells are washed with phosphate buffer saline (PBS) and the cells are fixed with 4% para formaldehyde at 4° C. for 30 minutes. Further, the 0.4% triton/PBS solution mixture is added and the cells are kept for 40 minutes. After 40 minutes the cells are washed with 0.1% PBS/tween three times. Further, 1% HSA/PBS solution mixture is added and the cells are kept in the mixture for 30 minutes. The cells are again subjected to 0.1% PBS/tween solution mixture. In the next step, the antibodies are blocked non-specifically with 1.5% goat serum, for 1 hour and incubated with initial antibodies and isotypes of them. Further, the cells are incubated with secondary rabbit antibody anti mouse IgG-FITC (at a dilution of 1:200; acbum, USA) for 4 hours. After incubation, the cells are washed with phosphaste buffer saline (PBS), and the inverted fluorescent microscope images are taken.

According to one embodiment herein, the quantitative real time PCR (qRT-PCR) is performed for the stem cells differentiated into ameloblast cells. For the qRT-PCR, the total RNA is extracted using the protocol of Rnasey PLUS mini kits. The PLUS RNX solution comprises of Tirozil, phenol and a strong detergent tissue for the tissue digestion and elimination of the cellular connections. The steps of RNA extraction comprises: adding 1 ml of the Rnasey PLUS mini kit solution to the cells samples. The samples are homogenized with an insulin syringe pipetting. 200 μl of the chloroform is added to the homogenized samples. The vials are incubated at 4° C. temperature for 60 minutes. The samples are centrifuged at 12000 rpm for 15 minutes at 4° C. After centrifugation, 1.5 ml of the supernatant is removed carefully and transferred to the tubes. A similar amount of cold isopropanol is added to the supernatant. The supernatant and the isopropanol are mixed to get a mixture and incubated in a refrigerator for 15 minutes. After incubation the mixture is centrifuged at 12000 rpm for 15 minutes at 4° C. The supernatant is discharged and 1 ml of ethanol is added in the pellet. The pellet is mixed with ethanol and centrifuged at 7500 rpm for 8 minutes at 4° C. The discharged supernatant is kept for 10 minutes under the hood for dry deposition of RNA. 35 μl Depc water is added to the total RNA, and transferred to −70° C.

According to one embodiment herein, the quantitative real time PCR (qRT-PCR) is performed for the stem cells differentiated into ameloblast cells. The cDNA is synthesized for the qRT-PCR. The purified RNA is taken from the freezer and added to a lyophilized tube. 20 ml of diethylpyrocarbonate (DEPC) is added to the RNA to de-ionize the water. The contents are completely dissolved by vortex and spin for a brief period. The cDNA synthesis of the mixture into vortex is carried out as follows: First connector primer is attached to RNA at 15-25° C. for 30 seconds. Secondly, the cDNA is synthesized at 42-45° C. for 4 minutes; Third, the RNA secondary structure patterns and the synthesized cDNA are melted at 55° C. for 30 seconds. These three stages of processes are repeated 12 times. Further the tube is incubated for 5 minutes at 95° C.

According to one embodiment herein, the cDNA is transferred to a temperature of −20° C. after the synthesis of the cDNA. The primers are designed for ameloblastin, amelogenin, cytokeratin 14 and amelotin. The gene sequences are extracted from http://www.ncbi.nlm.Nih site. Forward and reverse primers are generated using the software gene runner (version 3) and the primer express (version 3.05). The primers are prepared at a concentration from 100 pmol/ml to 10 pmol/ml.

According to one embodiment herein, 1 ml TRIzol® reagent is added to the RNA samples in a vial for the quantitative real time PCR (qRT-PCR) reaction. The RNA samples are then homogenized by pipetting multiple times using an insulin syringe as pipette. After homogenization, 200 μl of chloroform is added to all the samples. The vials are incubated at 4° C. temperature for 60 minutes. The incubated samples are centrifuged at 12000 rpm for 15 min at 4° C. The supernatant is removed carefully, and transferred precisely to the 1.5 ml tubes. 100 μl of cold isopropanol is added to the supernatant in the 1.5 ml tubes. The samples are centrifuged at 12000 rpm for 15 min at 4° C. The supernatant is discarded and 1 ml of ethanol is added to pellet. The precipitate is mixed and centrifuged at 7500 rpm for 8 min at 4° C. The discarded supernatant is kept for 10 minutes in the laminar flow cabinet for dry deposition. Next, 35 μl of RNase free water is added to the tube. The quality and quantity of the isolated RNA is examined by 260-280 nm absorption with spectrophotometer. The isolated RNA are transferred at 70° C. to refrigerator for storage.

According to one embodiment herein, the 1 μg RNA and oligo dT primer are added into the AccuPower RocketScript RT premix tubes for the cDNA synthesis. The volume is adjusted to a total volume of 20 μl with RNase free water and centrifuged several times. The reaction is performed under the following conditions: the mixture is subjected to a primer annealing process at 37° C., for 1 minute. The cDNA is synthesised at 70° C. for 60 minutes. The synthezised cDNA is subjected to heat Inactivation process at 95° C., for 5 minutes. The ameloblastin, amelogenin, cytokeratin 14, amelotin and B-action sequence are extracted from NCBI site for the real-time PCR reaction. The forward and the reverse primers are designed by using gene runner 3 and BeaconDesigner software. The forward primers (FW) and reverse primers (RV) concentration of each gene are adjusted to 10 pmol/ml.

| Code No | Company | Material |
|---|---|---|
| 667382 | Sigma | Tris (2,4-dimethyl-5-sulfophenyl) |
| | | phosphine trisodium salt |
| 352340 | Falcon | 40 micro m cell strainer |
| 352350 | Falcon | 70 micro m cell strainer |
| K-2044 | Bioneer | AccuPower ® RocketScript ™ |
| | | RT PreMix kit |
| h1758 | Sigma | HCl |
| a5533 | Sigma | Alizarin red |
| APF | Sigma | ALP assay kit |
| ab72776 | Abcam | Ameloblastin antibody |
| ab59705 | Abcam | Amelogenin antibody |
| A2942 | Sigma | Amphitericin B |
| 420042 | Sigma | Mgcl₂ |
| A2153 | Sigma | BSA |
| ab11415 | Abcam | CD105-antibody |
| orb10288 | Biorbyt | CD133-antibody |
| ab11415 | Abcam | CD146-antibody |
| 17100-017 | Invitrogen | Collagenase, type I |
| C9811-12EA | Sigma | CryoCane ™ |
| 21885025 | Invitrogen | DMEM low glucose with L- |
| | | glutamine (1 g/1) 500 mL |
| 31331028 | Invitrogen | DMEM/F-12 |
| d8418 | Sigma | DMSO |
| 10270106 | Invitrogen | FBS |
| F0291 | Sigma | FGF8 |
| 14170112 | Sigma | Hank's balanced salt solution 500 mL |
| H6136 | Sigma | Hanks media |
| 30412 | Sigma | HNa₂PO₄ |
| 25030-024 | Invitrogen | L-Glutamine |
| 000-10 | Yazd syringe | Syringe 10 cc |
| 000-05 | Yazd syringe | Syringe 5 cc |
| 000-02 | Yazd syringe | Syringe 2 cc |
| 204447 | Sigma | Sodium sulfate |
| 158127 | Sigma | paraformaldehyde |
| 421000013 | Sigma | Pipette |
| p7626 | Sigma | PMSF |
| 74136 | Qiagen | RNeasy ® PLUS mini kit |
| 59417C | Sigma | Trypsin/EDTA |
| BD | Sigma | Tube (l5 mL) |
| BD | Sigma | Tube (50 mL) |
| 90075 | Sigma | Flask T25 |
| 0002 | Sigma | Nitrate calcium |
| c1016 | Sigma | Cacl2 |
| 15596-026 | Invitrogen | TRIzol ® Reagent |
| 204143 | Qiagen | QuantiTect SYBR Green PCR Kit |
| K-2101 | Bioneer | AccuPower ® RocketScrpt ™ |
| | | RT PreMix |
| s3014 | Sigma | NaCl₂ |
| ab5758 | Abcam | Rabbit F(ab′) 2 Anti-Mouse IgG- |
| | | H&L (FITC), pre-adsorbed |
| | HBG (Germany) | Neobar lam cell counter |
| 14-9161-71 | eBioscience | Human Fc Receptor Binding |
| | | Inhibitor |
| | (Affymetrix) | Purified |
| D4818 | Sigma | Dispase I |

The embodiments herein introduce human endometrial stem cells (hEnSCs) as adult stem cells with the easy access source and no immunological response, for alternative cell therapy. The hEnSC were exposed to hepatogenic induction medium for 30 days. The ability of hEnSC to differentiate into Ameloblasts is examined through Co-culturing with the epithelial cells separated from embryonic tooth bud and FGF8. These cells are coaxed to oligodendrocyte progenitor differentiation by oligodendrocyte induction media and infection of these cells by miRNA219 and miRNA338 for 28 days. Differentiated cells are analyzed for expression of ameloblast markers by qRT-PCR and Immunocytochemistry. The results shows the expression of ameloblast cell markers. The EnSCs can response to the signaling molecules which usually used for ameloblast cell differentiation and for the first time as demonstrated herein that EnSCs is programmed to these cells and may convince to consider these cells as a unique source for liver repopulation and tooth reconstruction and cell therapy of neurodegenerative disease.

The differentiation of endometrial stem cells to Ameloblast cells is investigated by culturing rat embryonic tooth mesenchyme cells with growth factor FGF8. After 21 day the differentiated cells culturing expression of specific proteins called Amelogenin and Ameloblastin are investigated by cytochemistry. The protein expression level of Amelogenin, Ameloblastin and Enamelin and cytokeratin 14 measured by Quantitative Real-Time PCR. Immunocytochemistry and Real-Time PCR showed that the stem cells of endometrial are able to be differentiated into Ameloblast cells and express the tissue-specific Ameloblast proteins. Within 21 day treatment of cells in the differentiated culture, endometrial stem cells for expressing specific proteins of epithelial cells are induced and are differentiated to Ameloblast cells. In fact, in the present of appropriate odontogenic signals endometrial stem cells can be recommended as a useful source for dental tissue engineering which will be likely used in alternative treatments in future. This study shows that a bank of differentiated Ameloblast cells is used for commercialization while the ameloblast cells are used as produce natural product in dental tissue engineering and the treatment of dental diseases is performed with Ameloblast cells

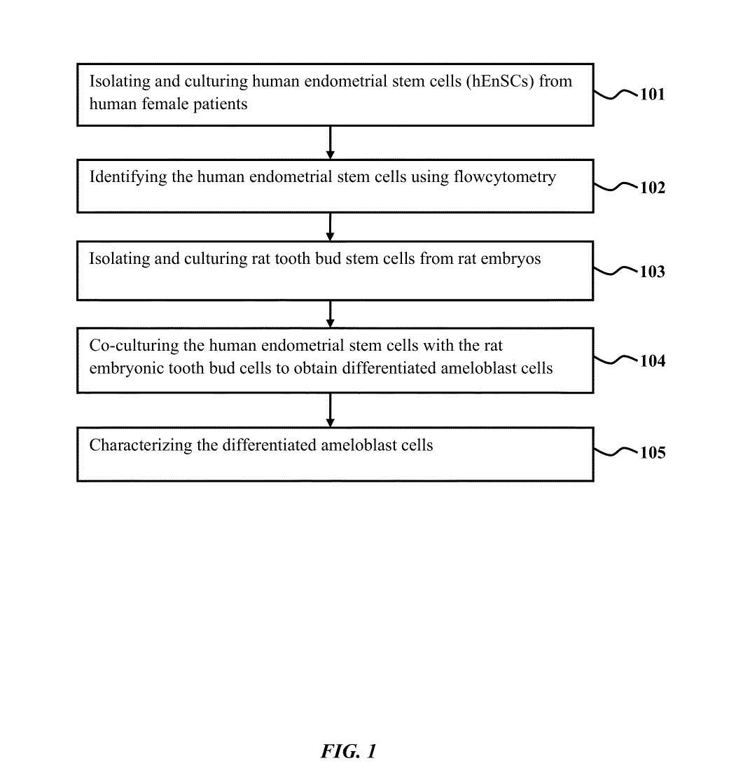

FIG. 1 illustrates a flow chart explaining the various steps carried out to obtain differentiated ameloblast cells from human endometrial stem cells and rat tooth embryonic cells, according to one embodiment herein. The human endometrial stem cells (hEnSCs) are isolated from human female patients and cultured (101). The human endometrial stem cells are identified using flow cytometry (102). The rat tooth bud stem cells are isolated from rat embryos and cultured (103). The human endometrial stem cells are co-cultured with the rat embryonic tooth bud cells to obtain differentiated ameloblast cells (104). The differentiated ameloblast cells are characterized (105).

The various embodiments herein provide a non-contact co-culture method for the differentiation of rat embryonic tooth bud and human endometrial stem cells (hEnSCs) to obtain ameloblast cells.

According to one embodiment herein, the co-culture method for the differentiation of rat embryonic tooth bud and human endometrial stem cells (hEnSCs) to obtain ameloblast cells comprises; isolating and culturing endometrial stem cells, isolating and culturing rat tooth bud mesenchymal cells, co-culturing the hEnSCs and rat tooth bud cells and characterization of the ameloblast cells by ARS, Immunochemical analysis and PCR techniques.

### Example 1.1

### Isolation of hEnSCs

Ten endometrial biopsy samples were obtained from female patients between the age groups of 18-40. The biopsy samples were transferred into Hanks fluid and washed in PBS containing antibiotics. The biopsy samples were sliced into small fragments and immersed in collagenase Type IA and 2 mg/mL of DMEM (containing antibiotics) for 2 hours at 37° C. Epithelial stem cells and stromal cells present in the endometrial biopsy were separated using 45 μm and 70 μm filters. These separated cells were then centrifuged at 1000 rpm for 15 minutes and purified by Ficoll purification. The purified cells were placed in a flask and incubated at 37° C. in 5% CO2 and 95% moisture. The purified stem cells were then analyzed using flow cytometry.

### Example 1.2

### Flow Cytometry Analysis of hEnSCs

Human endometrial stem cells were prepared for flow cytometry analysis by removing cell culture media from a cell plate. The cell plate was then rinsed once with 1×PBS. 5 mL of 0.2% EDTA (in PBS) was added to the cell plate. Cell surface staining was compromised when Trypsin/EDTA solution was used in the place of 0.2% EDTA. The hEnSCs were incubated in an incubator at 37° C. for 2 minutes. The hEnSCs were suspended in 5 mL of DMEM media that was added to neutralize EDTA. The suspended hEnSCs were collected in 15 mL tubes and centrifuged for 5 minutes at 1000 RPM. Prior to staining, the hEnSCs were transferred to the cell culture plate. 20 μL Human Fc Receptor Binding Inhibitor Purified was added (per 100 μL cell) to the culture plate. The culture plate was then incubated for 10-20 minutes at 2-8° C. or at room temperature. 50 μL of human endometrial cell suspension (from 2×105 to 108 cells) was aliquoted to each well in a culture plate. A recommended quantity of each primary antibody (CD90, anti-CD105, FITC-Phycoerythrin (PE), anti-CD 34, anti-human CD31, or PE-conjugated mouse IgG1 and anti CD61, anti CD14 all purchased from Abcam) were combined according to protocol to 1/1000 dilution of Flow cytometry Staining Buffer and added to each well. The final staining volume was 100 μL (50 μL of human endometrial cell sample+50 μL of antibody mix). This was then added to the human endometrial cell suspension with 105 cells/mL and gently vortexed. The hEnSCs were incubated for 30 minutes at 2-8° C. or on ice, to protect it from light. The hEnSCs were washed with 200 μL Flow cytometry Staining Buffer (per well). The hEnSCs were then centrifuged at 500 g for 5 minutes at room temperature. The hEnSCs formed a pellet. The centrifugation step was repeated for two washes hEnSCs were resuspended in an appropriate volume of Flow cytometry Staining Buffer. The prepared cells were then analyzed using flow cytometry.

### Example 1.3

### Isolation of Rat Embryonic Tooth Bud Cells

Eleven Wistar rat embryos aged 14 days were obtained from pregnant female rats that were anesthetized. The Rat mesenchymal tooth bud cells were isolated from the lower jaw of the rat embryo using a scalpel. The embryonic tooth bud tissues were placed in 1% trypsin at 4° C. for an enzymatic reaction called trypsinization. The trypsinization of rat embryonic tooth bud cells resulted in dental epithelial cells and mesenchymal cells. The dental epithelial cells and mesenchymal were completely separated under a stereo microscope. The dental epithelial and mesenchymal cells were rinsed in PBS and exposed to collagenase IA for 3 hours at 37° C. These dental epithelial and mesenchymal cells were then passed through a 70 mm Nylon filter, centrifuged at 2000 g for 15 min and rinsed again with PBS. The dental epithelial and mesenchymal cells were then transferred to a 6 well plate containing 0.5 mL DMEM culture medium (100× antibiotic, 10% FBS and 1% glutamine). The 6 well plate was incubated in 5% CO2 at 95% humidity. These dental epithelial and mesenchymal cells were maintained for a week to achieve a desired cell density. The growth medium was replaced once every 72 hours.

### Example 1.4

### Co-Culture of hEnSCs and Rat Embryonic Tooth Bud Cells

After the hEnSCs were grown for 3 passages (in order to reach a desired cell density of 1×106 cells/mL), the hEnSCs were transferred to culture plate inserts in a Home 6 well culture plate. The culture plate inserts contained 2 mL DMEM with 10% FBS culture medium. After rat mesenchymal tooth bud cells were grown for 2 passages (in order to reach a desired cell density of 1×105 cells/mL), the rat mesenchymal tooth bud cells were placed in the Home 6 well culture plate. After 24 hours, Fibroblast growth factor (Fgf8 at (100 ng/mv)) was added to the environment surrounding the endometrial cell. The hEnSCs and rat mesenchymal tooth bud cells were counted using a Neobar lam cell counter. The endometrial stem cells were treated for 14 days with differentiation medium and the medium was changed once every 72 hours.

### Example 1.5

### Checking the Morphological Characteristics of the Differentiated Cells

During the period of cell differentiation, morphological characteristics of the cells were observed regularly using an inverted microscope (Nikon, Japan TS-10).

### Example 1.6

### Assessing the Mineralized Nodule and Calcification by Alizarin Red Staining (ARS)

A mineralized nodule in an osteogenic cell culture provides a means of assessing mature osteoblast cell function and the status of culture. Formation of the mineralized nodule and calcification was analyzed in human endometrial stem cells after 28 days of co-culture. Alizarin Red Staining was used to stain the human endometrial stem cells. The hEnSCs were washed thrice in PBS and fixed in 4% formaldehyde for half an hour. The hEnSCs were then washed in PBS and stained with 2% ARS at pH 4.2-4.4 for 30 minutes at 37° C. The stained areas of the hEnSCs were observed and analyzed using an inverted microscope.

### Example 1.7

### Immunocytochemistry to Assess the Presence of Amelogenin and Ameloblastin in Differentiated Ameloblast Cells

Ameloblastin and amelogenin are enamel matrix protein that are expressed by differentiating ameloblast cells. After 14 days of human endometrial stem cells differentiation, Amelogenin (rabbit monoclonal anti-human) and ameloblastin (rabbit monoclonal anti-human) antibodies were used to assess the differentiation of hEnSCs into ameloblast cells. The human endometrial stem cells were washed with PBS and fixed with 4% paraformaldehyde at 4° C. for half an hour. 0.4% of Triton™ X-100 in PBS was added to the hEnSCs and incubated for 40 minutes. The hEnSCs were then washed thrice with PBS that containing 0.1% Tween® 20. PBS with 1% HSA was added to the hEnSCs after 30 minutes. The hEnSCs were washed once again with PBS containing 0.1% Tween® 20. Primary antibodies—Amelogenin and Ameloblastin antibodies were added to a culture plate containing hEnSCs. The primary antibodies were blocked nonspecifically with 1.5% goat serum. The culture plate was then incubated for 1 hour. Secondary Rabbit F(ab′)2 Anti-Mouse IgG—H&L (FITC), (at a dilution of 1:200) was then added to the same culture plate containing the primary antibodies and hEnSCs. The culture plate was then incubated for 4 hours. The secondary Rabbit F(ab′)2 Anti-Mouse IgG—H&L (FITC) was fluorescent in nature. The hEnSCs (with primary antibodies and fluorescent secondary antibodies) were visualized using an inverted fluorescent microscope.

### Example 1.8

### Total RNA Extraction for cDNA Synthesis

Total RNA was extracted from human endometrial stem cells after the hEnSCs were co-cultured with rat embryonic tooth bud cells. RNeasy® PLUS mini kit protocol was used to extract total RNA from the hEnSCs. RNX™-PLUS (containing Trizol, phenol and a strong detergent) was used for tissue digestion and elimination of cellular connections. 1 mL of RNeasy® PLUS mini kit solution was added to the hEnSCs and the cells were homogenized using an insulin syringe. 200 μL of chloroform was added to the vials containing the hEnSCs. The vials were incubated at 4° C. temperature for 60 minutes. The hEnSCs were centrifuged at 12000 RPM for 15 minutes at 4° C. The upper phase of the supernatant was carefully transferred to 1.5 mL tubes. Cold isopropanol solution of equal volume was added to the 1.5 ml tubes containing the upper phase of the supernatant. The hEnSCs were then mixed well and refrigerated for 15 minutes. The hEnSCs were then centrifuged at 12000 RPM for 15 minutes at 4° C. The supernatant formed was discarded. 1 ml of ethanol was added to the precipitate formed as a result of centrifugation. The precipitate and solution was then centrifuged at 7500 RPM for 8 minutes at 4° C. The supernatant formed after centrifugation was dried under a laminar hood for 10 minutes to form RNA deposits. The RNA deposits were suspended in 35 μL DEPC. The quality and quantity of isolated RNA was examined by 260/280 nm absorption with spectrophotometer. The RNA was then stored at −70° C.

### Example 1.9

### cDNA Synthesis

The cDNA was synthesized from the pure RNA using AccuPower® RocketScript™ RT PreMix. 1 μg of lyophilized purified RNA and oligo dT primer was added to an AccuPower® RocketScript™ RT PreMix tube. They volume in the tubes were adjusted to 20 μL with RNase free water. The tubes were vortexed several times. The reaction was performed under following conditions, for 12 cycles: The Primer is annealed at 37° C. for 1 minute. The cDNA is synthesised at 70° C. for 60 minutes. The heat inactivation of cDNA and RNA is performed at 95° C. for 5 minutes. The synthesized cDNA was stored at −20° C.

### Example 1.10

### Quantitative Real Time PCR (qRT-PCR) to Detect the Presence of Ameloblastin, Amelogenin, Amelotin and Cytokeratin 14 in Differentiated Ameloblast Cells

Primer sequences for Ameloblastin, Amelogenin, Cytokeratin 14 and Amelotin gene were extracted from NCBI site. Forward and reverse primers were designed by using Gene Runner 3 and Beacon Designer software. QuantiTect® SYBR® Green RT-PCR Kit (Qiagen) and Rotor Gene 6000 Real-Time PCR system were for qRT-PCR. The QuantiTect™ SYBR® Green PCR Kit contained HotStarTaq® DNA Polymerase, QuantiTect™ SYBR® Green PCR Buffer, dNTP mix including dUTP, SYBR® Green 1, ROX (passive reference dye), RNase free water and 5 mM MgCl. All human endometrial stem cell samples were diluted prior to qRT-PCR. The PCR master mix, forward and reverse primers were mixed properly and diluted based on the calculated values. The concentration of the forward primers (FW) and reverse primers (RV) for each gene was adjusted to 10 pmol/mL from an initial concentration of 100 pmol/ml. Table 1 shows sequences of Ameloblastin, Amelogenin and Cytokeratin 14 primers used for qRT-PCR. The reaction setup was carried out according to Table 2.

TABLE 1
Ameloblastin, Amelogenin, cytokeratin14 and
amelotin primer sequence

| | Type of | |
|---|---|---|
| Gene | primer | Primer sequences |
| Ameloblastin | F | —AACTATCTTGGTTGGCATCATCAG |
| Ameloblastin | R | —CCAGGAGGCATAGGATCAGTATC— |
| Amelogenin | F | —ACCTCTGCCTCCGATGTTCC— |
| Amelogenin | R | —TCCTCCCGCTTGGTCTTGTC— |
| Cytokeratin14 | F | —ACCTGAGCCGCATTCTGAAC— |
| Cytokeratin14 | R | —TCCACGCTGCCAATCATCTC— |
| Amelotin | F | —GCCTCATCATCCATTCCTTGTTC— |
| Amelotin | R | —AAGTCGTCATCTGTGCCACTG— |

TABLE 2
Temperature program used in Corbett 6000 Real Time PCR System

| Number of cycles | Temperature, Time | Steps |
|---|---|---|
| 1 | 95° C., 15 seconds | Primary Denaturing |
| 34-40 | 95° C., 5 seconds | Denaturation |
| | 60° C., 30 seconds | Annealing and extension |
| 1 | 95° C., 15 seconds | melting curve |
| | 60° C.-95° C., 30 seconds | |

### Example 2.1

### Isolation of hEnSCs

Ten endometrial biopsy samples were obtained from female patients between the age groups of 18-40. The biopsy samples were transferred into Hanks fluid and washed in PBS containing antibiotics. The biopsy samples were sliced into small fragments and immersed in trypsin and 2 mg/mL of DMEM (containing antibiotics) for 2 hours at 37° C. Epithelial stem cells and stromal cells present in the endometrial biopsy were separated using 45 μm and 70 μm filters. The separated cells were then centrifuged at 1000 rpm for 15 minutes and purified by Ficoll purification. The purified cells were placed in a flask and incubated at 37° C. in 5% CO2 and 95% moisture. The purified cells were then analyzed using flow cytometry.

### Example 2.2

### Flow Cytometry Analysis of hEnSCs

Human endometrial stem cells were prepared for flow cytometry analysis by removing cell culture media from a cell plate. The cell plate was then rinsed once with 1×PBS. 5 mL of 0.2% EDTA (in PBS) was added to the cell plate. Cell surface staining was compromised when Trypsin/EDTA solution was used in the place of 0.2% EDTA. The hEnSCs were incubated in an incubator at 37° C. for 2 minutes. The hEnSCs were suspended in 5 mL of DMEM media that was added to neutralize EDTA. The suspended hEnSCs were collected in 15 mL tubes and centrifuged for 5 minutes at 1000 RPM. Prior to staining, the hEnSCs were transferred to the cell culture plate. 20 μL Human Fc Receptor Binding Inhibitor Purified was added (per 100 μL cell) to the culture plate. The culture plate was then incubated for 10-20 minutes at 2-8° C. or at room temperature. 50 μL of human endometrial cell suspension (from 2×105 to 108 cells) was aliquoted to each well in a culture plate. A recommended quantity of each primary antibody (CD90, anti-CD105, FITC-Phycoerythrin (PE), anti-CD 34, anti-human CD31, or PE-conjugated mouse IgG1 and anti CD61, anti CD14 all purchased from Abcam) were combined according to protocol to 1/1000 dilution of Flow cytometry Staining Buffer and added to each well. The final staining volume was 100 μL (50 μL of human endometrial cell sample+50 μL of antibody mix). This was then added to the human endometrial cell suspension with 105 cells/mL and gently vortexed. The hEnSCs were incubated for 30 minutes at 2-8° C. or on ice, to protect it from light. The hEnSCs were washed with 200 μL Flow cytometry Staining Buffer (per well). The hEnSCs were then centrifuged at 500 g for 5 minutes at room temperature. The hEnSCs formed a pellet. The centrifugation step was repeated for two washes hEnSCs were resuspended in an appropriate volume of Flow cytometry Staining Buffer. The prepared cells were then analyzed using flow cytometry.

### Example 2.3

### Isolation of Rat Embryonic Tooth Bud Cells

Eleven Wistar rat embryos aged 7 days were obtained from pregnant female rats that were anesthetized. Rat mesenchymal tooth bud cells were isolated from the lower jaw of the rat embryo using a scalpel. The embryonic tooth bud tissues were incubated at 37° C. in 10 mM EDTA containing Ca2+, Mg2+ and PBS. The enzymatic reaction, lead to the formation of 2 separate cell populations-dental epithelial cells and mesenchymal cells. The dental epithelial cells and mesenchymal were completely separated under a stereo microscope. The dental epithelial and mesenchymal cells were rinsed in PBS and exposed to collagenase IA for 3 hours at 37° C. These dental epithelial and mesenchymal cells were then passed through a 70 mm Nylon filter, centrifuged at 1500 g for 15 min and rinsed again with PBS. The dental epithelial and mesenchymal cells were then transferred to a 6 well plate containing 0.5 mL DMEM culture medium (100× antibiotic, 10% FBS and 1% glutamine). The 6 well plate was incubated in 5% CO2 at 95% humidity. These dental epithelial and mesenchymal cells were maintained for a week to achieve a desired cell density. The growth medium was replaced once every 72 hours.

### Example 2.4

### Co-Culture of hEnSCs and Rat Embryonic Tooth Bud Cells

After the hEnSCs were grown for 3 passages (in order to reach a desired cell density of 1×106 cells/mL), the hEnSCs were transferred to culture plate inserts in a Home 6 well culture plate. The culture plate inserts contained 2 mL DMEM with 10% FBS culture medium. After rat mesenchymal tooth bud cells were grown for 2 passages (in order to reach a desired cell density of 1×105 cells/mL), the rat mesenchymal tooth bud cells were placed in the Home 6 well culture plate. After 24 hours, Bone morphogenetic protein (BMP2) (100 ng/mv) was added to the environment surrounding the endometrial cell. The hEnSCs and rat mesenchymal tooth bud cells were counted using a Neobar lam cell counter. The endometrial stem cells were treated for 14 days with differentiation medium and the medium was changed once every 72 hours.

### Example 2.5

### Checking the Morphological Characteristics of the Differentiated Cells

During the period of cell differentiation, morphological characteristics of the cells were observed regularly using an inverted microscope (Nikon, Japan TS-10).

### Example 2.6

### Assessing the Mineralized Nodule and Calcification by Alizarin Red Staining (ARS)

A mineralized nodule in an osteogenic cell culture provides a means of assessing mature osteoblast cell function and the status of culture. Formation of the mineralized nodule and calcification was analyzed in human endometrial stem cells after 28 days of co-culture. Alizarin Red Staining was used to stain the human endometrial stem cells. The hEnSCs were washed thrice in PBS and fixed in 4% formaldehyde for half an hour. The hEnSCs were then washed in PBS and stained with 2% ARS at pH 4.2-4.4 for 30 minutes at 37° C. The stained areas of the hEnSCs were observed and analyzed using an inverted microscope.

### Example 2.7

### Immunocytochemistry to Assess the Presence of Amelogenin and Ameloblastin in Differentiated Ameloblast Cells

Ameloblastin and amelogenin are enamel matrix protein that are expressed by differentiating ameloblast cells. After 14 days of human endometrial stem cells differentiation, Amelogenin (rabbit monoclonal anti-human) and ameloblastin (rabbit monoclonal anti-human) antibodies were used to assess the differentiation of hEnSCs into ameloblast cells. The human endometrial stem cells were washed with PBS and fixed with 4% paraformaldehyde at 4° C. for half an hour. 0.4% of Triton™ X-100 in PBS was added to the hEnSCs and incubated for 40 minutes. The hEnSCs were then washed thrice with PBS that containing 0.1% Tween® 20. PBS with 1% HSA was added to the hEnSCs after 30 minutes. The hEnSCs were washed once again with PBS containing 0.1% Tween® 20. Primary antibodies—Amelogenin and Ameloblastin antibodies were added to a culture plate containing hEnSCs. The primary antibodies were blocked nonspecifically with 1.5% goat serum. The culture plate was then incubated for 1 hour. Secondary Rabbit F(ab′)2 Anti-Mouse IgG—H&L (FITC), (at a 1:200 dilution) was then added to the same culture plate containing the primary antibodies and hEnSCs. The culture plate was then incubated for 4 hours. The secondary Rabbit F(ab′)2 Anti-Mouse IgG—H&L (FITC) was fluorescent in nature. The hEnSCs (with primary antibodies and fluorescent secondary antibodies) were visualized using an inverted fluorescent microscope.

### Example 2.8

### Total RNA Extraction for cDNA Synthesis

Total RNA was extracted from human endometrial stem cells after the hEnSCs were co-cultured with rat embryonic tooth bud cells. RNeasy® PLUS mini kit protocol was used to extract total RNA from the hEnSCs. RNX™-PLUS (containing Trizol, phenol and a strong detergent) was used for tissue digestion and elimination of cellular connections. 1 mL of RNeasy® PLUS mini kit solution was added to the hEnSCs and the cells were homogenized using an insulin syringe. 200 μL of chloroform was added to the vials containing the hEnSCs. The vials were incubated at 4° C. temperature for 60 minutes. The hEnSCs were centrifuged at 12000 RPM for 15 minutes at 4° C. The upper phase of the supernatant was carefully transferred to 1.5 mL tubes. Cold isopropanol solution of equal volume was added to the 1.5 ml tubes containing the upper phase of the supernatant. The hEnSCs were then mixed well and refrigerated for 15 minutes. The hEnSCs were then centrifuged at 12000 RPM for 15 minutes at 4° C. The supernatant formed was discarded. 1 ml of ethanol was added to the precipitate formed as a result of centrifugation. The precipitate and solution was then centrifuged at 7500 RPM for 8 minutes at 4° C. The supernatant formed after centrifugation was dried under a laminar hood for 10 minutes to form RNA deposits. The RNA deposits were suspended in 35 μL DEPC. The quality and quantity of isolated RNA was examined by 260/280 nm absorption with spectrophotometer. The RNA was then stored at −70° C.

### Example 2.9

### cDNA Synthesis

The cDNA was synthesized from the pure RNA using AccuPower® RocketScript™ RT PreMix. 1 μg of lyophilized purified RNA and oligo dT primer was added to an AccuPower® RocketScript™ RT PreMix tube. They volume in the tubes were adjusted to 20 μL with RNase free water. The tubes were vortexed several times. The reaction was performed under following conditions, for 12 cycles: The Primer is annealed at 37° C. for 1 minute. The cDNA is synthesised at 70° C. for 60 minutes. The heat inactivation of cDNA and RNA is performed at 95° C. for 5 minutes. The synthesized cDNA was stored at −20° C.

### Example 2.10

### Quantitative Real Time PCR (qRT-PCR) to Detect the Presence of Ameloblastin, Amelogenin, Amelotin and Cytokeratin 14 in Differentiated Ameloblast Cells

Primer sequences for Ameloblastin, Amelogenin, Cytokeratin 14 and Amelotin gene were extracted from NCBI site. Forward and reverse primers were designed by using Gene Runner 3 and Beacon Designer software. QuantiTect® SYBR® Green RT-PCR Kit (Qiagen) and Rotor Gene 6000 Real-Time PCR system were for qRT-PCR. The QuantiTect™ SYBR® Green PCR Kit contained HotStarTaq® DNA Polymerase, QuantiTect™ SYBR® Green PCR Buffer, dNTP mix including dUTP, SYBR® Green 1, ROX (passive reference dye), RNase free water and 5 mM MgCl. All human endometrial stem cell samples were diluted prior to qRT-PCR. The PCR master mix, forward and reverse primers were mixed properly and diluted based on the calculated values. The concentration of the forward primers (FW) and reverse primers (RV) for each gene was adjusted to 10 pmol/mL from an initial concentration of 100 pmol/ml. Table 1 shows sequences of Ameloblastin, Amelogenin and Cytokeratin 14 primers used for qRT-PCR. The reaction setup was carried out according to Table 2.

### Example 3.1

### Isolation of hEnSCs

Ten endometrial biopsy samples were obtained from female patients between the age groups of 18-40. The biopsy samples were transferred into Hanks fluid and washed in PBS containing antibiotics. The biopsy samples were sliced into small fragments and inserted into collagenase Type IA and 2 mg/mL of DMEM containing antibiotics for 2 hours at 37° C. Epithelial stem cells and stromal cells present in the endometrial biopsy were separated using 45 μm and 70 μm filters. These separated cells were then centrifuged at 1000 rpm for 15 minutes and purified by Ficoll purification. The purified cells were placed in a flask and incubated at 37° C. in 5% CO2 and 95% moisture. The purified stem cells were then analyzed using flow cytometry.

### Example 3.2

### Flow Cytometry Analysis of hEnSCs

Human endometrial stem cells were prepared for flow cytometry analysis by removing cell culture media from a cell plate. The cell plate was then rinsed once with 1×PBS. 5 mL of 0.2% EDTA (in PBS) was added to the cell plate. Cell surface staining was compromised when Trypsin/EDTA solution was used in the place of 0.2% EDTA. The hEnSCs were incubated in an incubator at 37° C. for 2 minutes. The hEnSCs were suspended in 5 mL of DMEM media that was added to neutralize EDTA. The suspended hEnSCs were collected in 15 mL tubes and centrifuged for 5 minutes at 1000 RPM. Prior to staining, the hEnSCs were transferred to the cell culture plate. 20 μL Human Fc Receptor Binding Inhibitor Purified was added (per 100 μL cell) to the culture plate. The culture plate was then incubated for 10-20 minutes at 2-8° C. or at room temperature. 50 μL of human endometrial cell suspension (from 2×105 to 108 cells) was aliquoted to each well in a culture plate. A recommended quantity of each primary antibody (CD90, anti-CD105, FITC-Phycoerythrin (PE), anti-CD 34, anti-human CD31, or PE-conjugated mouse IgG1 and anti CD61, anti CD14 all purchased from Abcam) were combined according to protocol to 1/1000 dilution of Flow cytometry Staining Buffer and added to each well. The final staining volume was 100 μL (50 μL of human endometrial cell sample+50 μL of antibody mix). This was then added to the human endometrial cell suspension with 105 cells/mL and gently vortexed. The hEnSCs were incubated for 30 minutes at 2-8° C. or on ice, to protect it from light. The hEnSCs were washed with 200 μL Flow cytometry Staining Buffer (per well). The hEnSCs were then centrifuged at 500 g for 5 minutes at room temperature. The hEnSCs formed a pellet. The centrifugation step was repeated for two washes hEnSCs were resuspended in an appropriate volume of Flow cytometry Staining Buffer. The prepared cells were then analyzed using flow cytometry.

### Example 3.3

### Isolation of Rat Embryonic Tooth Bud Cells

Eleven Wistar rat embryos aged 9 days were obtained from pregnant female rats that were anesthetized. Rat mesenchymal tooth bud tissues were isolated from the lower jaw of the rat embryo using a scalpel. The embryonic tooth bud cells were incubated at 4° C. for 2-3 hour in Dispase. The enzymatic reaction, lead to the formation of 2 separate cell populations—dental epithelial cells and mesenchymal cells. The dental epithelial cells and mesenchymal were completely separated under a stereo microscope. The dental epithelial and mesenchymal cells were rinsed in PBS and exposed to collagenase IA for 3 hours at 37° C. These dental epithelial and mesenchymal cells were then passed through a 70 mm Nylon filter, centrifuged at 2000 g for 15 min and rinsed again with PBS. The dental epithelial and mesenchymal cells were then transferred to a 6 well plate containing 0.5 mL RPMI culture medium (100× antibiotic, 10% FBS and 1% glutamine). The 6 well plate was incubated in 5% CO₂ at 95% humidity. These dental epithelial and mesenchymal cells were maintained for a week to achieve a desired cell density. The growth medium was replaced once every 72 hours.

### Example 3.4

### Co-Culture of hEnSCs and Rat Embryonic Tooth Bud Cells

The hEnSCs were grown for 3 passages (in order to reach a desired cell density of 1×106 cells/mL), the hEnSCs were transferred to culture plate inserts in a Home 6 well culture plate. The culture plate inserts contained 2 mL DMEM with 10% FBS culture medium. After rat mesenchymal tooth bud cells were grown for 2 passages (in order to reach a desired cell density of 1×105 cells/mL), the rat mesenchymal tooth bud cells were placed in the Home 6 well culture plate. After 24 hours, Bone morphogenetic protein (BMP4) (200 ng/mv) was added to the environment surrounding the endometrial cell. The hEnSCs and rat mesenchymal tooth bud cells were counted using a Neobar lam cell counter. The endometrial stem cells were treated for 14 days with differentiation medium and the medium was changed once every 72 hours.

### Example 3.5

### Checking the Morphological Characteristics of the Differentiated Cells

During the period of cell differentiation, morphological characteristics of the cells were observed regularly using an inverted microscope (Nikon, Japan TS-10).

### Example 3.6

### Assessing the Mineralized Nodule and Calcification by Alizarin Red Staining (ARS)

A mineralized nodule in an osteogenic cell culture provides a means of assessing mature osteoblast cell function and the status of culture. Formation of the mineralized nodule and calcification was analyzed in human endometrial stem cells after 28 days of co-culture. Alizarin Red Staining was used to stain the human endometrial stem cells. The hEnSCs were washed thrice in PBS and fixed in 4% formaldehyde for half an hour. The hEnSCs were then washed in PBS and stained with 2% ARS at pH 4.2-4.4 for 30 minutes at 37° C. The stained areas of the hEnSCs were observed and analyzed using an inverted microscope.

### Example 3.7

### Immunocytochemistry to Assess the Presence of Amelogenin and Ameloblastin in Differentiated Ameloblast Cells

Ameloblastin and amelogenin are enamel matrix protein that are expressed by differentiating ameloblast cells. After 14 days of human endometrial stem cells differentiation, Amelogenin (rabbit monoclonal anti-human) and ameloblastin (rabbit monoclonal anti-human) antibodies were used to assess the differentiation of hEnSCs into ameloblast cells. The human endometrial stem cells were washed with PBS and fixed with 4% paraformaldehyde at 4° C. for half an hour. 0.4% of Triton™ X-100 in PBS was added to the hEnSCs and incubated for 40 minutes. The hEnSCs were then washed thrice with PBS that containing 0.1% Tween® 20. PBS with 1% HSA was added to the hEnSCs after 30 minutes. The hEnSCs were washed once again with PBS containing 0.1% Tween® 20. Primary antibodies—Amelogenin and Ameloblastin antibodies were added to a culture plate containing hEnSCs. The primary antibodies were blocked nonspecifically with 1.5% goat serum. The culture plate was then incubated for 1 hour. Secondary Rabbit F(ab′)2 Anti-Mouse IgG—H&L (FITC), (at a 1:200 dilution) was then added to the same culture plate containing the primary antibodies and hEnSCs. The culture plate was then incubated for 4 hours. The secondary Rabbit F(ab′)2 Anti-Mouse IgG—H&L (FITC) was fluorescent in nature. The hEnSCs (with primary antibodies and fluorescent secondary antibodies) were visualized using an inverted fluorescent microscope.

### Example 3.8

### Total RNA Extraction for cDNA Synthesis

RNA was extracted from human endometrial stem cells after the hEnSCs were co-cultured with rat embryonic tooth bud cells. RNeasy® PLUS mini kit protocol was used to extract total RNA from the hEnSCs. RNX™-PLUS (containing Trizol, phenol and a strong detergent) was used for tissue digestion and elimination of cellular connections. 1 mL of RNeasy® PLUS mini kit solution was added to the hEnSCs and the cells were homogenized using an insulin syringe. 200 μL of chloroform was added to the vials containing the hEnSCs. The vials were incubated at 4° C. temperature for 60 minutes. The hEnSCs were centrifuged at 12000 RPM for 15 minutes at 4° C. The upper phase of the supernatant was carefully transferred to 1.5 mL tubes. Cold isopropanol solution of equal volume was added to the 1.5 ml tubes containing the upper phase of the supernatant. The hEnSCs were then mixed well and refrigerated for 15 minutes. The hEnSCs were then centrifuged at 12000 RPM for 15 minutes at 4° C. The supernatant formed was discarded. 1 ml of ethanol was added to the precipitate formed as a result of centrifugation. The precipitate and solution was then centrifuged at 7500 RPM for 8 minutes at 4° C. The supernatant formed after centrifugation was dried under a laminar hood for 10 minutes to form RNA deposits. The RNA deposits were suspended in 35 μL DEPC. The quality and quantity of isolated RNA was examined by 260/280 nm absorption with spectrophotometer. The RNA was then stored at −70° C.

### Example 3.9

### cDNA Synthesis

The cDNA was synthesized from the pure RNA using AccuPower® RocketScript™ RT PreMix. 1 μg of lyophilized purified RNA and oligo dT primer was added to an AccuPower® RocketScript™ RT PreMix tube. They volume in the tubes were adjusted to 20 μL with RNase free water. The tubes were vortexed several times. The reaction was performed under following conditions, for 12 cycles: The Primer is annealed at 37° C. for 1 minute. The cDNA is synthesised at 70° C. for 60 minutes. The heat inactivation of cDNA and RNA is performed at 95° C. for 5 minutes. The synthesized cDNA was stored at −20° C.

### Example 3.10

### Quantitative Real Time PCR (qRT-PCR) to Detect the Presence of Ameloblastin, Amelogenin, Amelotin and Cytokeratin 14 in Differentiated Ameloblast Cells

Primer sequences for Ameloblastin, Amelogenin, Cytokeratin 14 and Amelotin gene were extracted from NCBI site. Forward and reverse primers were designed by using Gene Runner 3 and Beacon Designer software. QuantiTect® SYBR® Green RT-PCR Kit (Qiagen) and Rotor Gene 6000 Real-Time PCR system were for qRT-PCR. The QuantiTect™ SYBR® Green PCR Kit contained HotStarTaq® DNA Polymerase, QuantiTect™ SYBR® Green PCR Buffer, dNTP mix including dUTP, SYBR® Green 1, ROX (passive reference dye), RNase free water and 5 mM MgCl. All human endometrial stem cell samples were diluted prior to qRT-PCR. The PCR master mix, forward and reverse primers were mixed properly and diluted based on the calculated values. The concentration of the forward primers (FW) and reverse primers (RV) for each gene was adjusted to 10 pmol/mL from an initial concentration of 100 pmol/ml. Table 1 shows sequences of Ameloblastin, Amelogenin and Cytokeratin 14 primers used for qRT-PCR. The reaction setup was carried out according to Table 2.

### Example 4.1

### Isolation of hEnSCs

Ten endometrial biopsy samples were obtained from female patients between the age groups of 18-40. The biopsy samples were transferred into Hanks fluid and washed in PBS containing antibiotics. The biopsy samples were sliced into small fragments and immerses in trypsin and 2 mg/mL of DMEM (containing antibiotics) for 2 hours at 37° C. Epithelial stem cells and stromal cells present in the endometrial biopsy were separated using 45 μm and 70 μm filters. These separated cells were then centrifuged at 1000 rpm for 15 minutes and purified by Ficoll purification. The purified cells were placed in a flask and incubated at 37° C. in 5% CO2 and 95% moisture. The purified stem cells were then analyzed using flow cytometry.

### Example 4.2

### Flow Cytometry Analysis of hEnSCs

Human endometrial stem cells were prepared for flow cytometry analysis by removing cell culture media from a cell plate. The cell plate was then rinsed once with 1×PBS. 5 mL of 0.2% EDTA (in PBS) was added to the cell plate. Cell surface staining was compromised when Trypsin/EDTA solution was used in the place of 0.2% EDTA. The hEnSCs were incubated in an incubator at 37° C. for 2 minutes. The hEnSCs were suspended in 5 mL of DMEM media that was added to neutralize EDTA. The suspended hEnSCs were collected in 15 mL tubes and centrifuged for 5 minutes at 1000 RPM. Prior to staining, the hEnSCs were transferred to the cell culture plate. 20 μL Human Fc Receptor Binding Inhibitor Purified was added (per 100 μL cell) to the culture plate. The culture plate was then incubated for 10-20 minutes at 2-8° C. or at room temperature. 50 μL of human endometrial cell suspension (from 2×105 to 108 cells) was aliquoted to each well in a culture plate. A recommended quantity of each primary antibody (CD90, anti-CD105, FITC-Phycoerythrin (PE), anti-CD 34, anti-human CD31, or PE-conjugated mouse IgG1 and anti CD61, anti CD14 all purchased from Abcam) were combined according to protocol to 1/1000 dilution of Flow cytometry Staining Buffer and added to each well. The final staining volume was 100 μL (50 μL of human endometrial cell sample+50 μL of antibody mix). This was then added to the human endometrial cell suspension with 105 cells/mL and gently vortexed. The hEnSCs were incubated for 30 minutes at 2-8° C. or on ice, to protect it from light. The hEnSCs were washed with 200 μL Flow cytometry Staining Buffer (per well). The hEnSCs were then centrifuged at 500 g for 5 minutes at room temperature. The hEnSCs formed a pellet. The centrifugation step was repeated for two washes hEnSCs were resuspended in an appropriate volume of Flow cytometry Staining Buffer. The prepared cells were then analyzed using flow cytometry.

### Example 4.3

### Isolation of Rat Embryonic Tooth Bud Cells

Eleven Wistar rat embryos aged 10 days were obtained from pregnant female rats that were anesthetized. Rat mesenchymal tooth bud tissues were isolated from the lower jaw of the rat embryo using a scalpel. The embryonic tooth bud cells were incubated at 4° C. for 2-3 hour in 1% trypsin. The enzymatic reaction, lead to the formation of 2 separate cell populations—dental epithelial cells and mesenchymal cells. The dental epithelial cells and mesenchymal were completely separated under a stereo microscope. The dental epithelial and mesenchymal cells were rinsed in PBS and exposed to collagenase IA for 3 hours at 37° C. These dental epithelial and mesenchymal cells were then passed through a 70 mm Nylon filter, centrifuged at 1000 g for 5 min and rinsed again with PBS. The dental epithelial and mesenchymal cells were then transferred to a 6 well plate containing 0.5 mL DMEM culture medium (100× antibiotic, 10% FCS and 1% glutamine). The 6 well plate was incubated in 5% CO₂ at 95% humidity. These dental epithelial and mesenchymal cells were maintained for a week to achieve a desired cell density. The growth medium was replaced once every 72 hours.

### Example 4.4

### Co-Culture of hEnSCs and Rat Embryonic Tooth Bud Cells

After the hEnSCs were grown for 3 passages (in order to reach a desired cell density of 1×106 cells/mL), the hEnSCs were transferred to culture plate inserts in a Home 6 well culture plate. The culture plate inserts contained 2 mL DMEM with 10% FBS culture medium. After rat mesenchymal tooth bud cells were grown for 2 passages (in order to reach a desired cell density of 1×105 cells/mL), the rat mesenchymal tooth bud cells were placed in the Home 6 well culture plate. After 24 hours, Fibroblast growth factor (Fgf8) (80 ng/mv) was added to the environment surrounding the endometrial cell. The hEnSCs and rat mesenchymal tooth bud cells were counted using a Neobar lam cell counter. The endometrial stem cells were treated for 14 days with differentiation medium and the medium was changed once every 72 hours.

### Example 4.5

### Checking the Morphological Characteristics of the Differentiated Cells

During the period of cell differentiation, morphological characteristics of the cells were observed regularly using an inverted microscope (Nikon, Japan TS-10).

### Example 4.6

### Assessing the Mineralized Nodule and Calcification by Alizarin Red Staining (ARS)

A mineralized nodule in an osteogenic cell culture provides a means of assessing mature osteoblast cell function and the status of culture. Formation of the mineralized nodule and calcification was analyzed in human endometrial stem cells after 28 days of co-culture. Alizarin Red Staining was used to stain the human endometrial stem cells. The hEnSCs were washed thrice in PBS and fixed in 4% formaldehyde for half an hour. The hEnSCs were then washed in PBS and stained with 2% ARS at pH 4.2-4.4 for 30 minutes at 37° C. The stained areas of the hEnSCs were observed and analyzed using an inverted microscope.

### Example 4.7

### Immunocytochemistry to Assess the Presence of Amelogenin and Ameloblastin in Differentiated Ameloblast Cells

Ameloblastin and amelogenin are enamel matrix protein that are expressed by differentiating ameloblast cells. After 14 days of human endometrial stem cells differentiation, Amelogenin (rabbit monoclonal anti-human) and ameloblastin (rabbit monoclonal anti-human) antibodies were used to assess the differentiation of hEnSCs into ameloblast cells. The human endometrial stem cells were washed with PBS and fixed with 4% paraformaldehyde at 4° C. for half an hour. 0.4% of Triton™ X-100 in PBS was added to the hEnSCs and incubated for 40 minutes. The hEnSCs were then washed thrice with PBS that containing 0.1% Tween® 20. PBS with 1% HSA was added to the hEnSCs after 30 minutes. The hEnSCs were washed once again with PBS containing 0.1% Tween® 20. Primary antibodies—Amelogenin and Ameloblastin antibodies were added to a culture plate containing hEnSCs. The primary antibodies were blocked nonspecifically with 1.5% goat serum. The culture plate was then incubated for 1 hour. Secondary Rabbit F(ab′)2 Anti-Mouse IgG—H&L (FITC), (at a 1:200 dilution) was then added to the same culture plate containing the primary antibodies and hEnSCs. The culture plate was then incubated for 4 hours. The secondary Rabbit F(ab′)2 Anti-Mouse IgG—H&L (FITC) was fluorescent in nature. The hEnSCs (with primary antibodies and fluorescent secondary antibodies) were visualized using an inverted fluorescent microscope.

### Example 4.8

### Total RNA Extraction for cDNA Synthesis

Total RNA was extracted from human endometrial stem cells after the hEnSCs were co-cultured with rat embryonic tooth bud cells. RNeasy® PLUS mini kit protocol was used to extract total RNA from the hEnSCs. RNX™-PLUS (containing Trizol, phenol and a strong detergent) was used for tissue digestion and elimination of cellular connections. 1 mL of RNeasy® PLUS mini kit solution was added to the hEnSCs and the cells were homogenized using an insulin syringe. 200 μL of chloroform was added to the vials containing the hEnSCs. The vials were incubated at 4° C. temperature for 60 minutes. The hEnSCs were centrifuged at 12000 RPM for 15 minutes at 4° C. The upper phase of the supernatant was carefully transferred to 1.5 mL tubes. Cold isopropanol solution of equal volume was added to the 1.5 ml tubes containing the upper phase of the supernatant. The hEnSCs were then mixed well and refrigerated for 15 minutes. The hEnSCs were then centrifuged at 12000 RPM for 15 minutes at 4° C. The supernatant formed was discarded. 1 ml of ethanol was added to the precipitate formed as a result of centrifugation. The precipitate and solution was then centrifuged at 7500 RPM for 8 minutes at 4° C. The supernatant formed after centrifugation was dried under a laminar hood for 10 minutes to form RNA deposits. The RNA deposits were suspended in 35 μL DEPC. The quality and quantity of isolated RNA was examined by 260/280 nm absorption with spectrophotometer. The RNA was then stored at −70° C.

### Example 4.9

### cDNA Synthesis

The cDNA was synthesized from the pure RNA using AccuPower® RocketScript™ RT PreMix. 1 μg of lyophilized purified RNA and oligo dT primer was added to an AccuPower® RocketScript™ RT PreMix tube. They volume in the tubes were adjusted to 20 μL with RNase free water. The tubes were vortexed several times. The reaction was performed under following conditions, for 12 cycles: The Primer is annealed at 37° C. for 1 minute. The cDNA is synthesised at 70° C. for 60 minutes. The heat inactivation of cDNA and RNA is performed at 95° C. for 5 minutes. The synthesized cDNA was stored at −20° C.

### Example 4.10

### Quantitative Real Time PCR (qRT-PCR) to Detect the Presence of Ameloblastin, Amelogenin, Amelotin and Cytokeratin 14 in Differentiated Ameloblast Cells

Primer sequences for Ameloblastin, Amelogenin, Cytokeratin 14 and Amelotin gene were extracted from NCBI site. Forward and reverse primers were designed by using Gene Runner 3 and Beacon Designer software. QuantiTect® SYBR® Green RT-PCR Kit (Qiagen) and Rotor Gene 6000 Real-Time PCR system were for qRT-PCR. The QuantiTect™ SYBR® Green PCR Kit contained HotStarTaq® DNA Polymerase, QuantiTect™ SYBR® Green PCR Buffer, dNTP mix including dUTP, SYBR® Green 1, ROX (passive reference dye), RNase free water and 5 mM MgCl. All human endometrial stem cell samples were diluted prior to qRT-PCR. The PCR master mix, forward and reverse primers were mixed properly and diluted based on the calculated values. The concentration of the forward primers (FW) and reverse primers (RV) for each gene was adjusted to 10 pmol/mL from an initial concentration of 100 pmol/ml. Table 1 shows sequences of Ameloblastin, Amelogenin and Cytokeratin 14 primers used for qRT-PCR. The reaction setup was carried out according to Table 2.

### Example 5.1

### Isolation of hEnSCs

Ten endometrial biopsy samples were obtained from female patients between the age groups of 18-40. The biopsy samples were transferred into Hanks fluid and washed in PBS containing antibiotics. The biopsy samples were sliced into small fragments and immersed in trypsin and 2 mg/mL of DMEM (containing antibiotics) for 2 hours at 37° C. Epithelial stem cells and stromal cells present in the endometrial biopsy were separated using 45 μm and 70 μm filters. The separated cells were then centrifuged at 1000 rpm for 15 minutes and purified by Ficoll purification. The purified cells were placed in a flask and incubated at 37° C. in 5% CO2 and 95% moisture. The purified cells were then analyzed using flow cytometry.

### Example 5.2

### Flow Cytometry Analysis of hEnSCs

Human endometrial stem cells were prepared for flow cytometry analysis by removing cell culture media from a cell plate. The cell plate was then rinsed once with 1×PBS. 5 mL of 0.2% EDTA (in PBS) was added to the cell plate. Cell surface staining was compromised when Trypsin/EDTA solution was used in the place of 0.2% EDTA. The hEnSCs were incubated in an incubator at 37° C. for 2 minutes. The hEnSCs were suspended in 5 mL of DMEM media that was added to neutralize EDTA. The suspended hEnSCs were collected in 15 mL tubes and centrifuged for 5 minutes at 1000 RPM. Prior to staining, the hEnSCs were transferred to the cell culture plate. 20 μL Human Fc Receptor Binding Inhibitor Purified was added (per 100 μL cell) to the culture plate. The culture plate was then incubated for 10-20 minutes at 2-8° C. or at room temperature. 50 μL of human endometrial cell suspension (from 2×105 to 108 cells) was aliquoted to each well in a culture plate. A recommended quantity of each primary antibody (CD90, anti-CD105, FITC-Phycoerythrin (PE), anti-CD 34, anti-human CD31, or PE-conjugated mouse IgG1 and anti CD61, anti CD14 all purchased from Abcam) were combined according to protocol to 1/1000 dilution of Flow cytometry Staining Buffer and added to each well. The final staining volume was 100 μL (50 μL of human endometrial cell sample+50 μL of antibody mix). This was then added to the human endometrial cell suspension with 105 cells/mL and gently vortexed. The hEnSCs were incubated for 30 minutes at 2-8° C. or on ice, to protect it from light. The hEnSCs were washed with 200 μL Flow cytometry Staining Buffer (per well). The hEnSCs were then centrifuged at 500 g for 5 minutes at room temperature. The hEnSCs formed a pellet. The centrifugation step was repeated for two washes hEnSCs were resuspended in an appropriate volume of Flow cytometry Staining Buffer. The prepared cells were then analyzed using flow cytometry.

### Example 5.3

### Isolation of Rat Embryonic Tooth Bud Cells

Eleven Wistar rat embryos aged 12 days were obtained from pregnant female rats that were anesthetized. Rat mesenchymal tooth bud cells were isolated from the lower jaw of the rat embryo using a scalpel. The embryonic tooth bud tissues were placed in Collagenase IA for 2-3 hours at 4° C., for an enzymatic digestion. The enzymatic digestion of rat embryonic tooth bud cells resulted in dental epithelial cells and mesenchymal cells. The dental epithelial cells and mesenchymal were completely separated under a stereo microscope. The dental epithelial and mesenchymal cells were rinsed in PBS and exposed to Dispase for 3 hours at 37° C. These dental epithelial and mesenchymal cells were then passed through a 70 mm Nylon filter, centrifuged at 2000 g for 15 min and rinsed again with PBS. The dental epithelial and mesenchymal cells were then transferred to a 6 well plate containing 0.5 mL DMEM culture medium (100× antibiotic, 10% FBS and 1% glutamine). The 6 well plate was incubated in 5% CO2 at 95% humidity. These dental epithelial and mesenchymal cells were maintained for a week to achieve a desired cell density. The growth medium was replaced once every 72 hours.

### Example 5.4

### Co-Culture of hEnSCs and Rat Embryonic Tooth Bud Cells

The hEnSCs were grown for 3 passages (in order to reach a desired cell density of 1×10⁶ cells/mL), the hEnSCs were transferred to culture plate inserts in a Home 6 well culture plate. The culture plate inserts contained 2 mL DMEM with 10% FBS culture medium. After rat mesenchymal tooth bud cells were grown for 2 passages (in order to reach a desired cell density of 1×10⁵ cells/mL), the rat mesenchymal tooth bud cells were placed in the Home 6 well culture plate. After 24 hours, Fibroblast growth factor (Fgf8 (100 ng/mv)) was added to the environment surrounding the endometrial cell. The hEnSCs and rat mesenchymal tooth bud cells were counted using a Neobar lam cell counter. The endometrial stem cells were treated for 14 days with differentiation medium and the medium was changed once every 72 hours.

### Example 5.5

### Checking the Morphological Characteristics of the Differentiated Cells

During the period of cell differentiation, morphological characteristics of the cells were observed regularly using an inverted microscope (Nikon, Japan TS-10).

### Example 5.6

### Assessing the Mineralized Nodule and Calcification by Alizarin Red Staining (ARS)

A mineralized nodule in an osteogenic cell culture provides a means of assessing mature osteoblast cell function and the status of culture. Formation of the mineralized nodule and calcification was analyzed in human endometrial stem cells after 28 days of co-culture. Alizarin Red Staining was used to stain the human endometrial stem cells. The hEnSCs were washed thrice in PBS and fixed in 4% formaldehyde for half an hour. The hEnSCs were then washed in PBS and stained with 2% ARS at pH 4.2-4.4 for 30 minutes at 37° C. The stained areas of the hEnSCs were observed and analyzed using an inverted microscope.

### Example 5.7

### Immunocytochemistry to Assess the Presence of Amelogenin and Ameloblastin in Differentiated Ameloblast Cells

Ameloblastin and amelogenin are enamel matrix protein that are expressed by differentiating ameloblast cells. After 14 days of human endometrial stem cells differentiation, Amelogenin (rabbit monoclonal anti-human) and ameloblastin (rabbit monoclonal anti-human) antibodies were used to assess the differentiation of hEnSCs into ameloblast cells. The human endometrial stem cells were washed with PBS and fixed with 4% paraformaldehyde at 4° C. for half an hour. 0.4% of Triton™ X-100 in PBS was added to the hEnSCs and incubated for 40 minutes. The hEnSCs were then washed thrice with PBS that containing 0.1% Tween® 20. PBS with 1% HSA was added to the hEnSCs after 30 minutes. The hEnSCs were washed once again with PBS containing 0.1% Tween® 20. Primary antibodies—Amelogenin and Ameloblastin antibodies were added to a culture plate containing hEnSCs. The primary antibodies were blocked nonspecifically with 1.5% goat serum. The culture plate was then incubated for 1 hour. Secondary Rabbit F(ab′)2 Anti-Mouse IgG—H&L (FITC), (at a 1:200 dilution) was then added to the same culture plate containing the primary antibodies and hEnSCs. The culture plate was then incubated for 4 hours. The secondary Rabbit F(ab′)2 Anti-Mouse IgG—H&L (FITC) was fluorescent in nature. The hEnSCs (with primary antibodies and fluorescent secondary antibodies) were visualized using an inverted fluorescent microscope.

### Example 5.8

### Total RNA Extraction for cDNA Synthesis

Total RNA was extracted from human endometrial stem cells after the hEnSCs were co-cultured with rat embryonic tooth bud cells. RNeasy® PLUS mini kit protocol was used to extract total RNA from the hEnSCs. RNX™-PLUS (containing Trizol, phenol and a strong detergent) was used for tissue digestion and elimination of cellular connections. 1 mL of RNeasy® PLUS mini kit solution was added to the hEnSCs and the cells were homogenized using an insulin syringe. 200 μL of chloroform was added to the vials containing the hEnSCs. The vials were incubated at 4° C. temperature for 60 minutes. The hEnSCs were centrifuged at 12000 RPM for 15 minutes at 4° C. The upper phase of the supernatant was carefully transferred to 1.5 mL tubes. Cold isopropanol solution of equal volume was added to the 1.5 ml tubes containing the upper phase of the supernatant. The hEnSCs were then mixed well and refrigerated for 15 minutes. The hEnSCs were then centrifuged at 12000 RPM for 15 minutes at 4° C. The supernatant formed was discarded. 1 ml of ethanol was added to the precipitate formed as a result of centrifugation. The precipitate and solution was then centrifuged at 7500 RPM for 8 minutes at 4° C. The supernatant formed after centrifugation was dried under a laminar hood for 10 minutes to form RNA deposits. The RNA deposits were suspended in 35 μL DEPC. The quality and quantity of isolated RNA was examined by 260-280 nm absorption with spectrophotometer. The RNA was then stored at −70° C.

### Example 5.9

### cDNA Synthesis

The cDNA was synthesized from the pure RNA using AccuPower® RocketScript™ RT PreMix. 1 μg of lyophilized purified RNA and oligo dT primer was added to an AccuPower® RocketScript™ RT PreMix tube. They volume in the tubes were adjusted to 20 μL with RNase free water. The tubes were vortexed several times. The reaction was performed under following conditions, for 12 cycles: The Primer is annealed at 37° C. for 1 minute. The cDNA is synthesised at 70° C. for 60 minutes. The heat inactivation of cDNA and RNA is performed at 95° C. for 5 minutes. The synthesized cDNA was stored at −20° C.

### Example 5.10

### Quantitative Real Time PCR (qRT-PCR) to Detect the Presence of Ameloblastin, Amelogenin, Amelotin and Cytokeratin 14 in Differentiated Ameloblast Cells

Primer sequences for Ameloblastin, Amelogenin, Cytokeratin 14 and Amelotin gene were extracted from NCBI site. Forward and reverse primers were designed by using Gene Runner 3 and Beacon Designer software. QuantiTect® SYBR® Green RT-PCR Kit (Qiagen) and Rotor Gene 6000 Real-Time PCR system were for qRT-PCR. The QuantiTect™ SYBR® Green PCR Kit contained HotStarTaq® DNA Polymerase, QuantiTect™ SYBR® Green PCR Buffer, dNTP mix including dUTP, SYBR® Green 1, ROX (passive reference dye), RNase free water and 5 mM MgCl. All human endometrial stem cell samples were diluted prior to qRT-PCR. The PCR master mix, forward and reverse primers were mixed properly and diluted based on the calculated values. The concentration of the forward primers (FW) and reverse primers (RV) for each gene was adjusted to 10 pmol/mL from an initial concentration of 100 pmol/ml. Table 1 shows sequences of Ameloblastin, Amelogenin and Cytokeratin 14 primers used for qRT-PCR. The reaction setup was carried out according to Table 2.

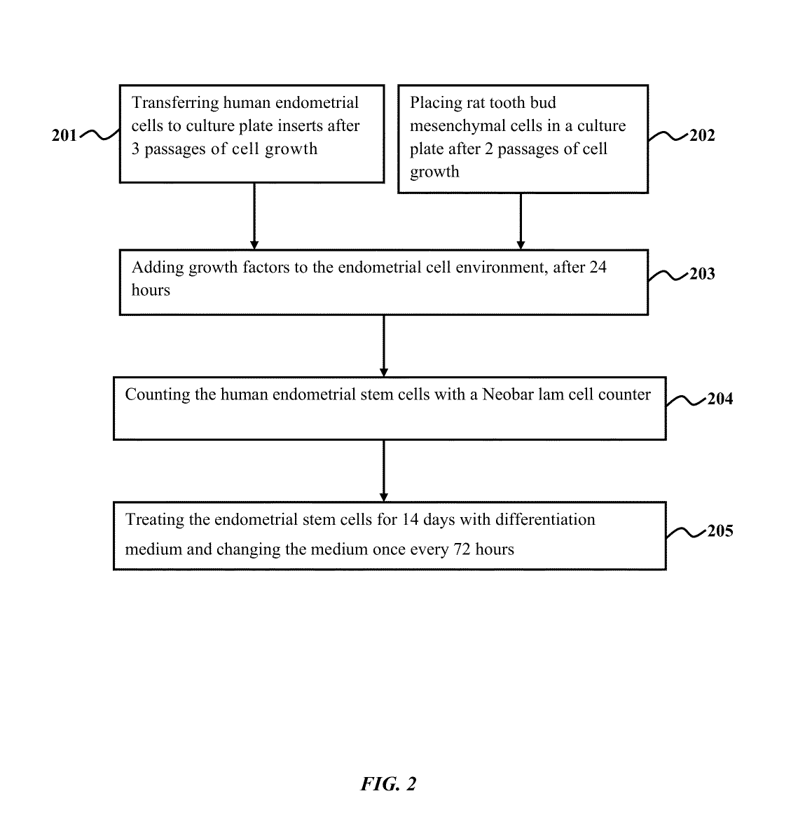

FIG. 2 illustrates a flow chart explaining the various steps involved in co-culturing the human endometrial stem cells and rat tooth embryonic cells to obtain ameloblast cells, according to one embodiment herein. The human endometrial cells are transferred to culture plate inserts after 3 passages of cell growth (201). The rat tooth bud mesenchymal cells are placed in a culture plate after 2 passages of cell growth (202). Growth factors are added to the endometrial cell environment, after 24 hours (203). The human endometrial stem cells are counted with a Neobar lam cell counter (204). The endometrial stem cells are treated for 14 days with differentiation medium and the medium is changed once in every 72 hours (205).

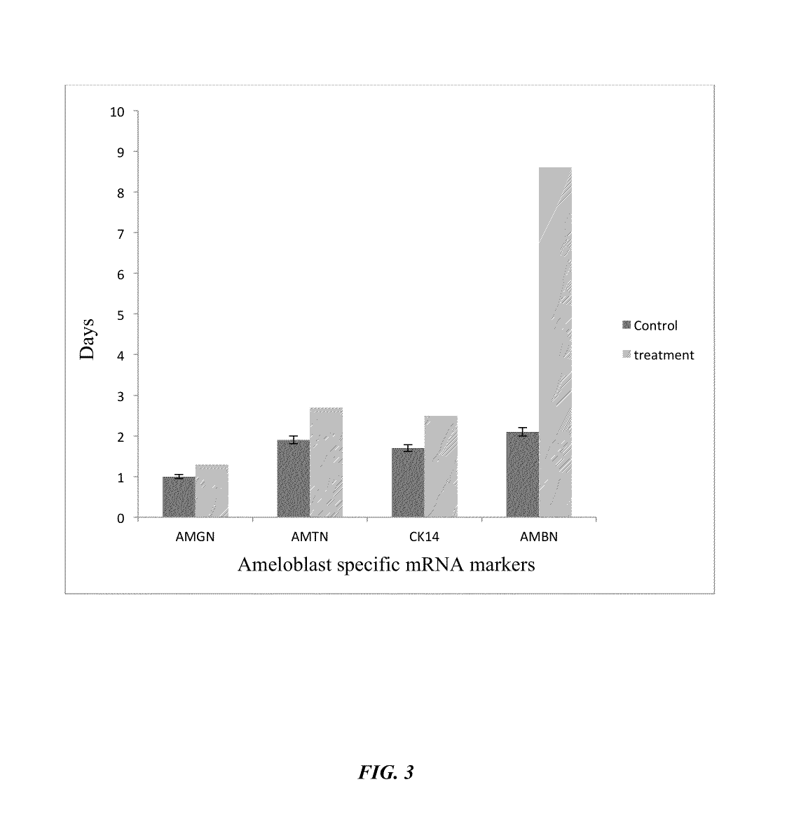

FIG. 3 illustrates a histogram exhibiting a relative gene expression of ameloblast specific mRNA markers, according to one embodiment herein. Quantitative real time PCR is used to identify the expression of ameloblast specific mRNA markers in differentiated and undifferentiated hEnSCs on day 14 of the hEnSCs induction. The specific ameloblast specific mRNA markers are CK14, AMTN, AMGN and AMBN. The hEnSCs cultured at basal conditions is used as a control in this study. P-value is P<0.05, ***P<0.001. Error bars show ±SEM, n=3 samples. The GAPDH is used as a housekeeping gene control. The GAPDH is one of the most commonly used housekeeping genes used in comparisons of gene expression data.

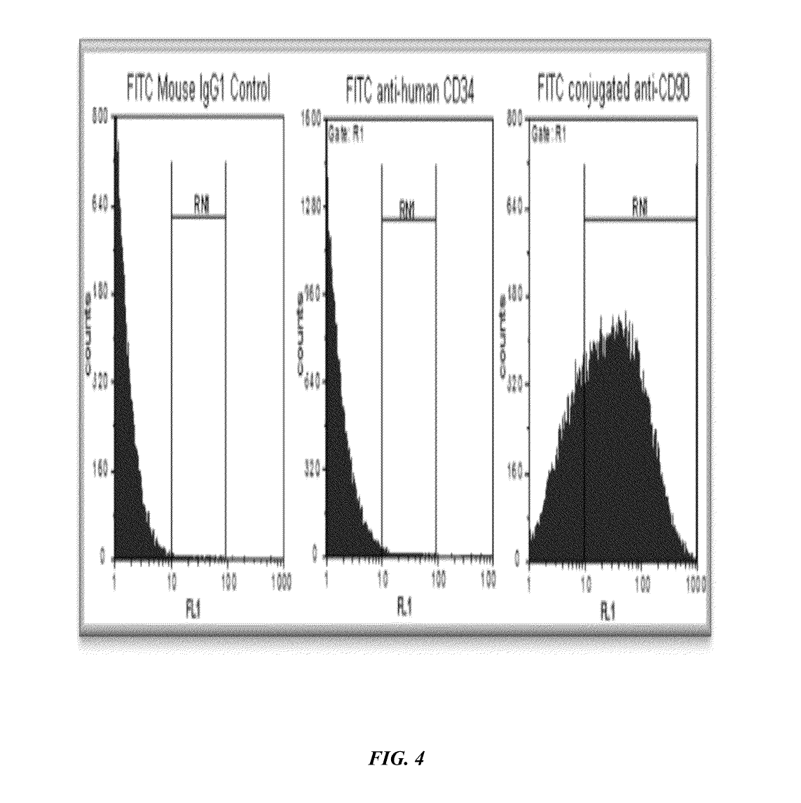

FIG. 4 illustrates a histogram indicting a flow cytometry analysis of endometrial cells for mesenchymal stem cell marker CD90 and hematopoietic stem cell marker CD34, according to one embodiment herein. The histogram shows the negative expression of CD34 and positive expression of CD90 in human endometrial stem cells. CD34 is a member of a family of single-pass trans membrane proteins that is expressed on early hematopoietic and vascular-associated tissue. The expression of CD34 in mesenchymal hEnSCs should be negative. CD90 is expressed on the surface of a variety of non-lymphoid tissues and mesenchymal stromal cells. The expression of CD90 in mesenchymal hEnSCs should be positive.

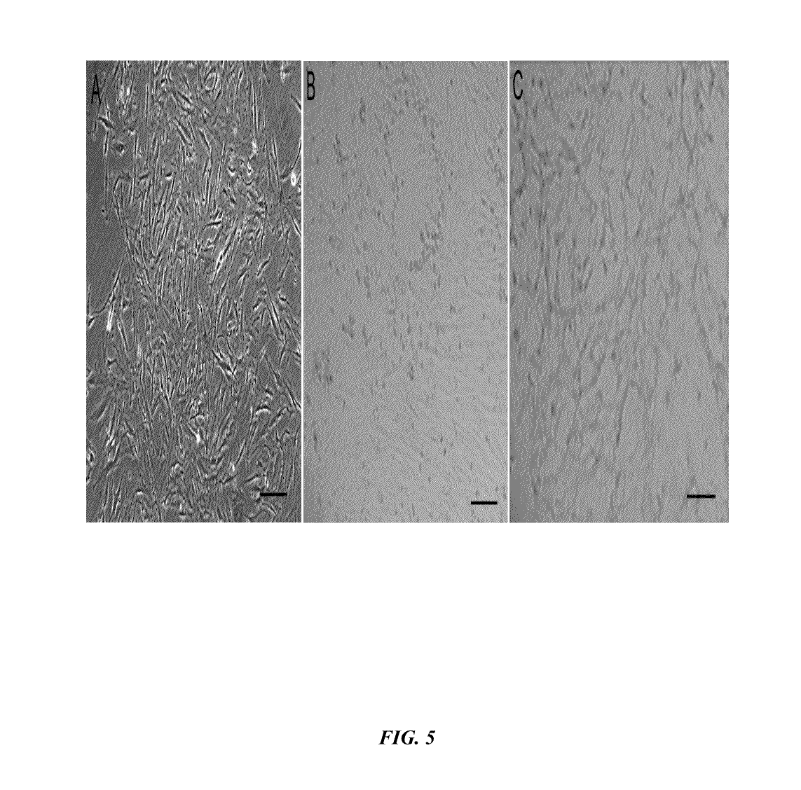

FIG. 5A-5C illustrate an image indicating the morphological characteristics of human endometrial stem cells before and after differentiation, according to one embodiment herein. FIG. 5A is an image showing the morphological characteristics of human endometrial stem cells before differentiation. FIG. 5B is an image showing the morphological characteristics of human endometrial stem cells after 4 days of differentiation FIG. 5C is an image showing the morphological characteristics of human endometrial stem cells after 14 days differentiation.

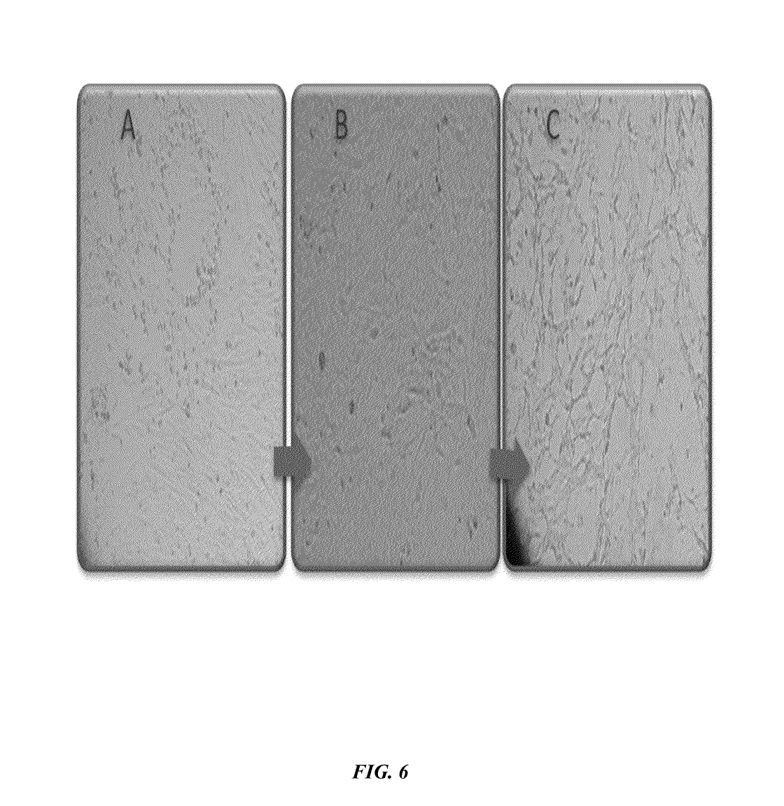

FIG. 6A-6C illustrate an image indicating the morphological characteristics of differentiated human endometrial stem cells (hEnSCs) after co-culturing the hEnSCs with rat embryo dental tooth bud stem cells, according to one embodiment herein. Differentiated cells become polarized and as a result of their shape, function and structure changes. Polarity enables a cell to carry out specialized functions. FIG. 6A is an image indicating the morphological characteristics of differentiated human endometrial stem cells after seven days of co-culturing with rat embryonic dental tooth bud stem cells. FIG. 6B illustrates an image indicating the morphological characteristics of differentiated human endometrial stem cells after twenty days of co-culturing with rat embryonic dental tooth bud stem cells. FIG. 6C illustrates an image indicating the morphological characteristics of differentiated human endometrial stem cells after twenty eight days of co-culturing with rat embryonic dental tooth bud stem cells. The control cells remain undifferentiated without any change in morphological characteristics.

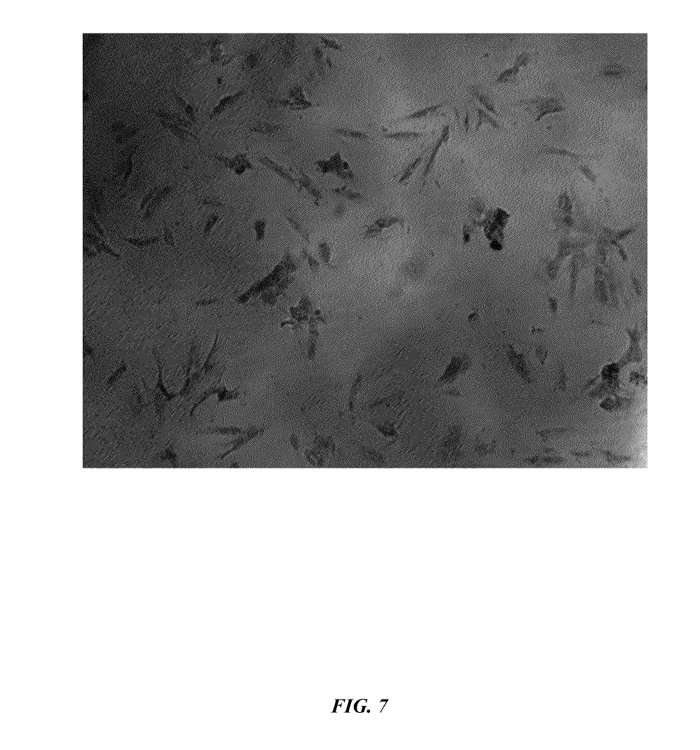

FIG. 7 illustrates an image indicating the mineralized nodule stained with Alizarin red stain, according to one embodiment herein. A mineralized nodule in an osteogenic cell culture provides a means of assessing mature osteoblast cell function and the status of culture. FIG. 7 illustrates an image indicating the mineralized nodule stained with Alizarin red stain. The image is taken 14 days after human endometrial stem cells are co-cultured with rat embryonic tooth bud stem cells. No mineralized nodule is observed in the control group of cells.

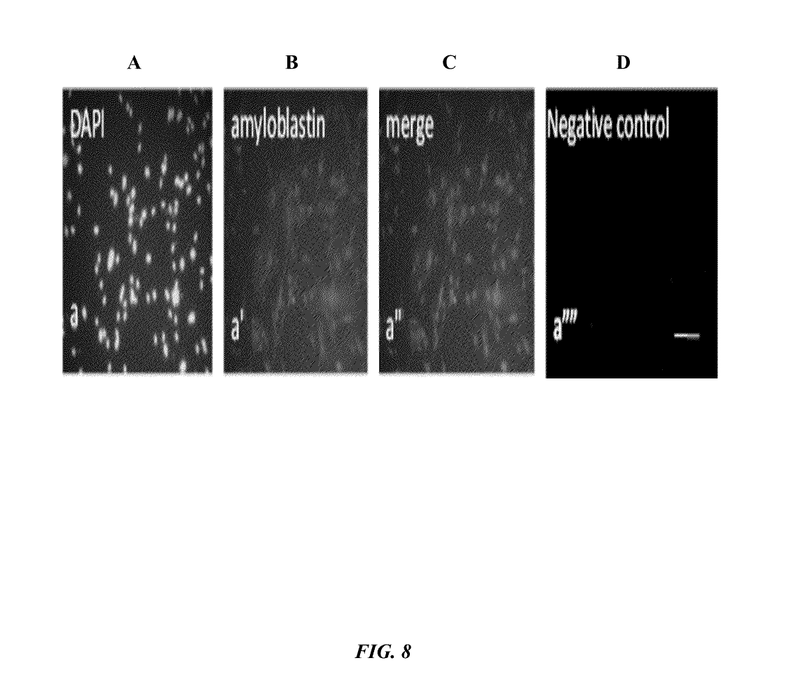

FIG. 8A-8D illustrate an image indicating the immunocytochemical analysis of Ameloblastin markers, in the differentiated human endometrial stem cells (hEnSCs), according to one embodiment herein. Immunocytochemical analysis is used to assess the presence of Ameloblastin in differentiated ameloblast cells. Ameloblastin is an enamel matrix protein that is expressed by differentiating ameloblast cells. The 4′,6-diamidino-2-phenylindole (DAPI) is a fluorescent stain that binds strongly to the A-T rich regions in DNA. DAPI is used extensively in the fluorescence microscopy. FIG. 8A illustrates an image of DAPI stain alone. FIG. 8B illustrate an image of Ameloblastin marker alone. FIG. 8C illustrate an image of Ameloblastin marker stained with DAPI stain. The immunocytochemical analysis of the hEnSCs Ameloblastin marker stained with the DAPI shows the presence of differentiated ameloblast cells. FIG. 8D illustrate an image of negative control cells (with no Ameloblastin markers) stained with DAPI. Since there are no Ameloblastin markers present on the negative control cells, the negative control cells have not differentiated into ameloblast cells.

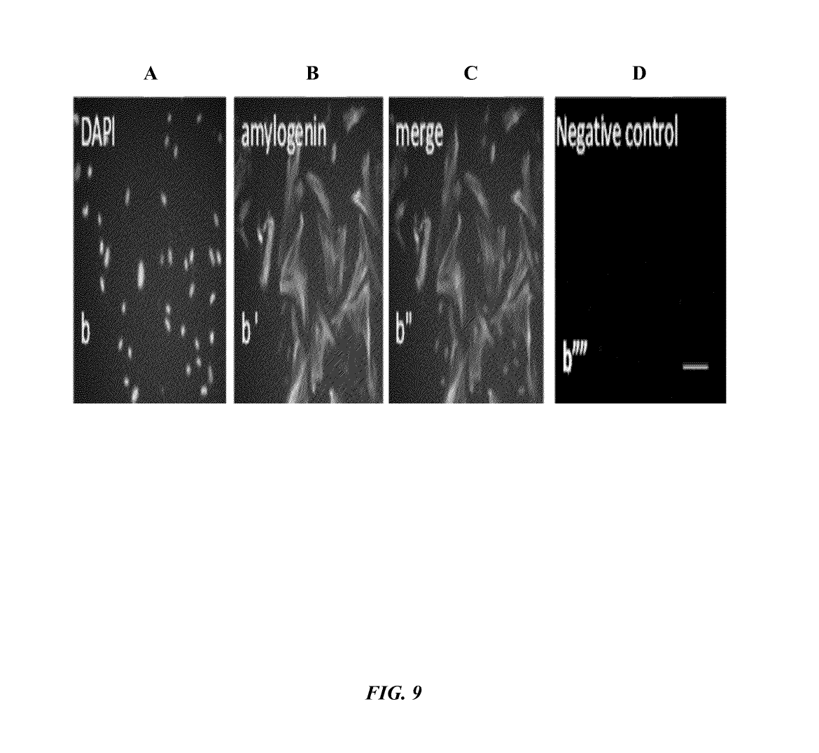

FIG. 9A-9D illustrate an image indicating the immunocytochemical analysis of Amelogenin markers, in the differentiated human endometrial stem cells (hEnSCs), according to one embodiment herein. Immunocytochemistry is used to assess the presence of Amelogenin in differentiated ameloblast cells. Amelogenin is an enamel matrix protein that is expressed by differentiating ameloblast cells. The 4′,6-diamidino-2-phenylindole (DAPI) is a fluorescent stain that binds strongly to the A-T rich regions in DNA. The DAPI is used extensively in fluorescence microscopy. FIG. 9A is an image of DAPI stain alone. FIG. 9B is an image of Amelogenin marker alone. FIG. 9C is an image of Amelogenin marker stained with DAPI stain. The immunocytochemical analysis of the hEnSCs Amelogenin marker stained with DAPI, shows the presence of differentiated ameloblast cells. FIG. 9D is an image of negative control cells (with no Amelogenin markers) stained with DAPI. Since there are no Amelogenin markers present on the negative control cells, this shows that the negative control cells have not differentiated into ameloblast cells.

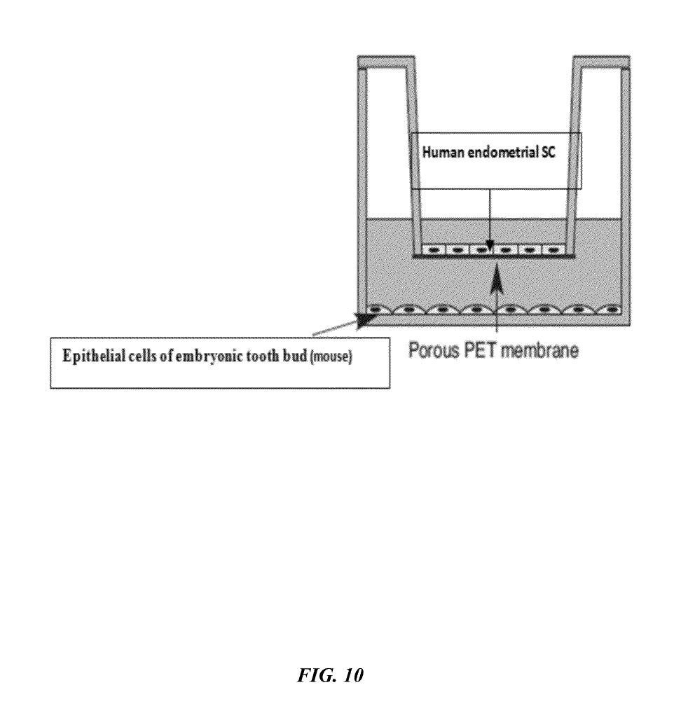

FIG. 10 illustrates a schematic representation of a non-contact co-culture system. The non contact co-culture system comprises of a culture plate insert and a culture plate. Human endometrial stem cells (hEnSCs) (mesenchymal cells) are placed in the culture plate insert and rat embryonic tooth bud cells (epithelial cells) are placed in the culture plate. The culture plate insert separates the hEnSCs from the rat embryonic tooth bud cells. During co-culture the rat embryonic tooth bud cells (epithelial cells) in the culture plate undergo an epithelial-mesenchymal transition (EMT). An epithelial-mesenchymal transition (EMT) is a biologic process that allows a polarized epithelial cell, which normally interacts with basement membrane via its basal surface, to undergo multiple biochemical changes that enable it to assume a mesenchymal cell phenotype, which includes enhanced migratory capacity, invasiveness and an elevated resistance to apoptosis. The completion of an EMT is signaled by the degradation of underlying basement membrane and the formation of a mesenchymal cell that can migrate away from the epithelial layer in which it originated. The rat embryonic tooth bud cells that undergoes an EMT, interacts with the human endometrial mesenchymal stem cells stem cells via a paracrine signaling. The paracrine signaling is a form of cell-cell communication in which a cell produces a signal to induce changes in nearby cells, altering the behavior or differentiation of those cells. Signaling molecules known as paracrine factors diffuse over a relatively short distance (local action). This paracrine signaling from the rat embryonic tooth bud cells (now mesenchymal) helps in the differentiation of human endometrial stem cells to the ameloblast cells.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method of synthesizing ameloblast cells, the method comprising steps of:
isolating and culturing human endometrial stem cells (hEnSCs) from human female patients;
identifying the human endometrial stem cells using flow cytometry;
isolating and culturing rat tooth bud stem cell from rat embryos;
co-culturing the human endometrial stem cells with the rat embryonic tooth bud cells to obtain differentiated ameloblast cells; and
characterizing the differentiated ameloblast cells.

2. The method according to claim 1, wherein step of isolating and culturing human endometrial stem cells (hEnSCs), comprising steps of:
acquiring a plurality of endometrial biopsy samples from female patients of preset age group, and wherein the female patients are in the age groups of 18-40;
transferring the endometrial biopsy samples into Hanks fluid;
washing the endometrial biopsy samples in a phospahte buffer saline (PBS) containing antibiotics;
slicing the endometrial biopsy samples into small fragments;
immersing the small fragments into a proteolytic enzymes and DMEM containing antibiotics for two hours at 37° C., and wherein the proteolyitc enzyme is selected from a group consisting of a collagenase IA and a trypsin;
separating the epithelial stem cells and the stromal cells present in the endometrial biopsy samples using a plurality of filters, and wherein the plurality of filters has a size of 45 μm and 70 μm;
centrifuging the separated cells at 1000 rpm for 15 minutes and purifying the cells by a Ficoll purification;
placing the purified cells in a flask and incubating the purified cells at 37° C. in an atmosphere containing 5% CO₂ and 95% moisture; and
analyzing the purified cells using a flow cytometry.

3. The method according to claim 1, wherein isolating and culturing the rat embryonic tooth bud cells, comprising steps of:
acquiring a plurality of Wistar rat embryos from an anaesthetized pregnant female rat, wherein the acquired rat embryos are in an age group of 7 days, 9 days, 10 days, 12 days and 14 days;
isolating the rat mesenchymal tooth bud tissue from a lower jaw of the rat embryo using a scalpel;
trypsinizing the embryonic tooth bud tissue, by placing the tooth bud tissue in 1% trypsin at 4° C. and wherein trypsinization of the rat embryo tooth bud tissue yields dental epithelial cells and mesenchymal cells;
separating a dental epithelium cells and a mesenchymal cells under a stereo microscope;
rinsing the dental epithelial cells and the mesenchymal cells in phosphate buffer saline (PBS);
subjecting the dental epithelial cells and the mesenchymal cells to an enzyme for 3 hours at 37° C.;
filtering the dental epithelial cells and the mesenchymal cells using a nylon filter with a pore size of 70 mm and obtaining a filtrate;
centrifuging the filtrate at 2000 g for 15 minutes and rinsing the centrifuged filtrate with phosphate buffer saline;
transferring the dental epithelial cells and mesenchymal cells to a culture plate containing 0.5 mL DMEM culture medium, and wherein the DMEM culture medium further comprises 100× antibiotic, 10% FBS and 1% gentamycine, and wherein the well culture plate is a 6 well culture plate;
incubating the well culture plate in an atmosphere containing 5% CO₂, 95% humidity for two weeks; and
replacing the growth medium once every 72 hours.

4. The method according to claim 3, wherein the enzyme is selected from a group consisting of collagenase IA, trypsin, dispase and a chelating agent and wherein the chelating agent comprises 10 mM EDTA with Ca²⁺, Mg²⁺, and PBS.

5. The method according to claim 3, wherein the rat mesenchymal tooth bud tissues with different ages are treated with different chelating agent or enzyme, and wherein the 7 days old rat mesenchymal tooth bud tissue is treated with a chelating agent comprising 10 mM EDTA containing Ca²⁺, Mg²⁺ and PBS, and wherein the 9 days old rat mesenchymal tooth bud tissue is treated with an enzyme dispase, and wherein the 10 days old rat mesenchymal tooth bud tissue is treated with an enzyme trypsin, and wherein the 12 days old rat mesenchymal tooth bud tissue is treated with an enzyme collagenase IA, and wherein the 14 days old rat mesenchymal tooth bud tissue is treated with an enzyme trypsin.

6. The method according to claim 1, wherein step of co-culturing the human endometrial stem cells (hEnSCs) with the rat embryonic tooth bud cells to obtain a differentiated ameloblast cells comprising steps of:
culturing the human endometrial stem cells (hEnSCs) for three passages; wherein the hEnScs reach a desired cell density of 1×10⁶ cells/ml;
transferring the hEnSCs to a culture plate inserts of the 6 well culture plate, wherein the culture plate insert comprises 2 mL DMEM with 10% FBS culture medium;
culturing the rat mesenchymal tooth bud cells for two passages, wherein the rat mesenchymal tooth bud cells are cultured for obtaining a cell density of 1×10⁵ cells/ml;
transferring the rat mesenchymal tooth bud cells in the 6 well culture plate;
putting the culture insert with hEnSCs in the culture plate containing rat mesenchymal tooth bud cells;
adding a growth factor agent to an environment surrounding the endometrial cells;
counting the hEnSCs ad the rat mesenchymal tooth bud cells using a neobar lam cell counter;
treating the endometrial stem cells with differentiation medium; and
changing the medium once in every 72 hours.

7. The method according to claim 6, wherein the growth factor agent is selected from a group consisting of a Fgf8, a BMP2 and a BMP4.

8. The method according to claim 6, wherein the rat embryonic mesenchymal tooth bud cells of different ages are co-cultured with the human endometrial stem cells (hEnSCs) supplemented with different growth factor agents, and wherein the 14 days old rat embryonic mesenchymal tooth bud cells are co-cultured with the human endometrial stem cells (hEnSCs) supplemented with a growth factor agent of Fgf8, and wherein the 7 days old rat embryonic mesenchymal tooth bud cells are co-cultured with the human endometrial stem cells (hEnSCs) supplemented with a growth factor agent of BMP2, and wherein the 9 days old rat embryonic mesenchymal tooth bud cells are co-cultured with the human endometrial stem cells (hEnSCs) supplemented with a growth factor agent of BMP4, and wherein the 10 days old rat embryonic mesenchymal tooth bud cells are co-cultured with the human endometrial stem cells (hEnSCs) supplemented with a growth factor agent of Fgf8, and wherein the 12 days old rat embryonic mesenchymal tooth bud cells are co-cultured with the human endometrial stem cells (hEnSCs) supplemented with a growth factor agent of Fgf8.
